(19) United States
(12) Patent Application Publication
(10) Pub. No.: US 2016/0338608 A1
(43) Pub. Date: Nov. 24, 2016

(54) MAGNETIC MEASUREMENT SYSTEM

(71) Applicant: SEIKO EPSON CORPORATION, Tokyo (JP)

(72) Inventors: Kimio NAGASAKA, Hokuto-shi (JP); Mitsutoshi MIYASAKA, Suwa-shi (JP)

(73) Assignee: SEIKO EPSON CORPORATION, Tokyo (JP)

(21) Appl. No.: 15/157,929

(22) Filed: May 18, 2016

(30) Foreign Application Priority Data
May 22, 2015 (JP) 2015-104275

(51) Int. Cl. A61B 5/04; A61B 5/00

(52) U.S. Cl. CPC A61B 5/04007; A61B 5/04008; A61B 5/7225; A61B 5/7203; G01R 33/0023

(57) ABSTRACT

A magnetic measurement system includes a heart magnetic field sensor that measures a first magnetic field and a second magnetic field, a noise magnetic sensor that measures the second magnetic field, and a magnetic measurement apparatus that computes an approximate value of the second magnetic field in the heart magnetic field sensor by using a measurement value in the noise magnetic sensor and a multi-variable polynomial. The magnetic measurement apparatus subtracts the approximate value of the second magnetic field from a measurement value in the heart magnetic field sensor.

### BACKGROUND

1. Technical Field

The present invention relates to a magnetic measurement system.

2. Related Art

A magnetic measurement apparatus for measuring a magnetic field of the heart or a magnetic field of the brain, weaker than terrestrial magnetism, has been proposed (for example, refer to JP-A-5-297087). The magnetic field measurement apparatus is noninvasive, and can measure states of organs without applying a load to a subject (living body). JP-A-5-297087 discloses a living body magnetic measurement apparatus having a configuration in which a sensor (pickup coil) measuring a magnetic field generated from a living body, and a sensor (reference coil) measuring an environmental magnetic field acting as noise are provided, and an environmental magnetic field (noise) included in a magnetic field measured by the pickup coil is removed on the basis of an environmental magnetic field measured by the reference coil.

In the living body magnetic measurement apparatus disclosed in JP-A-5-297087, the number of reference coils is smaller than the number of pickup coils, and an environmental magnetic field at a position of each pickup coil is obtained on the basis of data measured by reference coils disposed at positions corresponding to some of the pickup coils. In addition, environmental magnetic fields at positions of other pickup coils at which corresponding reference coils are not disposed are obtained through linear interpolation and estimation by using data measured by the reference coils.

In the living body magnetic measurement apparatus disclosed in JP-A-5-297087, the reference coil is disposed at a position which is different (separated) from a position where the pickup coil is disposed. For this reason, an environmental magnetic field obtained on the basis of the data measured by the reference coil does not necessarily match an environmental magnetic field at the position of the pickup coil. An environmental magnetic field at the position of the pickup coil where a corresponding reference coil is not disposed is estimated on the basis of the data measured by the reference coil. A magnetic field generated from a living body is obtained by subtracting the measurement data and the estimation data of the environmental magnetic field from the measurement data at the position of the pickup coil. Therefore, there is a concern that an error tends to occur in data obtained as an environmental magnetic field at the position of the pickup coil, and thus a magnetic field generated from a living body cannot be measured with high accuracy.

### SUMMARY

An advantage of some aspects of the invention is to solve the problems described above, and the invention can be implemented as the following aspects or application examples.

### Application Example 1

A magnetic measurement system according to this application example includes a first magnetic sensor that measures a first magnetic field and a second magnetic field; a second magnetic sensor that measures the second magnetic field; and a processing apparatus that computes an approximate value of the second magnetic field in the first magnetic sensor by using a measurement value in the second magnetic sensor and a multi-variable polynomial.

According to the configuration of this application example, the processing apparatus computes an approximate value of the second magnetic field in the first magnetic sensor by using a measurement value of the second magnetic field measured by the second magnetic sensor and a multi-variable polynomial. Thus, it is possible to compute, with high accuracy, an approximate value of the second magnetic field in the first magnetic sensor measuring the first magnetic field and the second magnetic field.

### Application Example 2

A magnetic measurement system according to this application example includes a first magnetic sensor that measures a first magnetic field and a second magnetic field; a second magnetic sensor that measures the second magnetic field; and a processing apparatus that computes an approximate value of the second magnetic field in the first magnetic sensor by using a measurement value in the second magnetic sensor and a non-linear polynomial.

According to the configuration of this application example, the processing apparatus computes an approximate value of the second magnetic field in the first magnetic sensor by using a measurement value of the second magnetic field measured by the second magnetic sensor and a non-linear polynomial. Thus, it is possible to compute, with high accuracy, an approximate value of the second magnetic field in the first magnetic sensor measuring the first magnetic field and the second magnetic field.

### Application Example 3

In the magnetic measurement system according to the application example, it is preferable that the processing apparatus subtracts the approximate value of the second magnetic field from a measurement value in the first magnetic sensor.

According to the configuration of this application example, the processing apparatus subtracts the approximate value of the second magnetic field in the first magnetic sensor from a measurement value in the first magnetic sensor measuring the first magnetic field and the second magnetic field, and thus the first magnetic field measured by the first magnetic sensor is obtained. The approximate value of the second magnetic field is computed with high accuracy on the basis of the measurement value in the second magnetic sensor, and thus it is possible to calculate the first magnetic field with high accuracy.

### Application Example 4

In the magnetic measurement system according to the application example, it is preferable that the multi-variable polynomial is expressed by Equation (1).

Bᵢ=aᵢ₁+aᵢ₂x+aᵢ₃y+aᵢ₄z+aᵢ₅xy+aᵢ₆yz+aᵢ₇zx (1)

In Equation (1), aᵢⱼ (where i is an integer of 1 to 3, and j is an integer of 1 to 7) is a coefficient, x, y, and z are space coordinates of an approximate value B of a magnetic field, and Bᵢ is an i-th component of the approximate value B of the magnetic field.

According to the present inventor's intensive examination, a magnetic field vector at any position in a measurement target space has proved to be approximated with high accuracy by using the multi-variable polynomial shown in Equation (1). The first term aᵢ₁ of the right side of Equation (1) indicates a parallel magnetic field in the entire space, the second term aᵢ₂x to the fourth term aᵢ₄y indicate a linear magnetic field, and the fifth term aᵢ₅xy to the seventh term aᵢ₇zx indicate an alternating magnetic field (torsion of the magnetic field). A component which is proportional to an outer product term between a current element vector and a position vector is present in a magnetic field according to the Biot-Savart law. Therefore, the present inventor has introduced an xy term, a yz term, and a zx term representing torsion from a fifth term to a seventh term into an approximate expression of the magnetic field. According to the configuration of this application example, an approximate value of the second magnetic field is computed by using the multi-variable polynomial shown in Equation (1), and thus it is possible to compute the approximate value of the second magnetic field in the first magnetic sensor with high accuracy.

### Application Example 5

In the magnetic measurement system according to the application example, it is preferable that the non-linear polynomial is expressed by the above Equation (1).

According to the configuration of this application example, an approximate value of the second magnetic field is computed by using the non-linear polynomial shown in Equation (1), and thus it is possible to compute the approximate value of the second magnetic field in the first magnetic sensor with high accuracy.

### Application Example 6

In the magnetic measurement system according to the application example, it is preferable that a solution of the polynomial is obtained by using a least square method on the basis of the measurement value in the second magnetic sensor.

According to the configuration of this application example, since a solution of Equation (1) is obtained by using the least square method on the basis of the measurement value in the second magnetic sensor, and an approximate value of the second magnetic field is computed, it is possible to calculate the approximate value of the second magnetic field with high accuracy.

### Application Example 7

In the magnetic measurement system according to the application example, it is preferable that the second magnetic sensor measures 21 or more magnetic field vector components of the second magnetic field.

According to the configuration of this application example, in the multi-variable polynomial or the non-linear polynomial shown in Equation (1), 7 unknowns are present for each of XYZ components, and thus a total of 21(=3×7) unknowns are present. Therefore, 21 or more magnetic field vector components of the second magnetic field are measured, and thus it is possible to compute an approximate value of the second magnetic field with high accuracy by using Equation (1).

### Application Example 8

In the magnetic measurement system according to the application example, it is preferable that, when a first matrix formed of unknowns of the above Equation (1) is indicated by a which is expressed by Equation (2), a second matrix formed of the measurement value in the second magnetic sensor is indicated by M which is expressed by Equation (3), and a third matrix formed of a position of the second magnetic sensor is indicated by P which is expressed by Equation (4), the first matrix a is obtained by using Equation (5) or Equation (6).

$$\begin{matrix}\vec{B} ( \vec{r} ) = ( \begin{matrix}{B}_{x} \\ {B}_{y} \\ {B}_{z}\end{matrix} ) = ( \begin{matrix}{a}_{11} & {a}_{12} & {a}_{13} & {a}_{14} & {a}_{15} & {a}_{16} & {a}_{17} \\ {a}_{21} & {a}_{22} & {a}_{23} & {a}_{24} & {a}_{25} & {a}_{26} & {a}_{27} \\ {a}_{31} & {a}_{32} & {a}_{33} & {a}_{34} & {a}_{35} & {a}_{36} & {a}_{37}\end{matrix} ) ( \begin{matrix}1 \\ x \\ y \\ z \\ xy \\ yz \\ xz\end{matrix} ) ≡ a \vec{R} & ( 2 ) \\ M = ( {\vec{B}}_{1} ⋯ {\vec{B}}_{α} ) = ( \begin{matrix}{B}_{11} & {B}_{12} & … & {B}_{1 α} \\ {B}_{21} & {B}_{22} & … & {B}_{2 α} \\ {B}_{31} & {B}_{32} & … & {B}_{3 α}\end{matrix} ) & ( 3 ) \\ P = ( {\vec{R}}_{1} ⋯ {\vec{R}}_{α} ) = ( \begin{matrix}{R}_{11} & {R}_{12} & … & {R}_{1 α} \\ {R}_{21} & {R}_{22} & … & {R}_{2 α} \\ {R}_{31} & {R}_{32} & … & {R}_{3 α} \\ {R}_{41} & {R}_{42} & … & {R}_{4 α} \\ {R}_{51} & {R}_{52} & … & {R}_{5 α} \\ {R}_{61} & {R}_{62} & … & {R}_{6 α} \\ {R}_{71} & {R}_{72} & … & {R}_{7 α}\end{matrix} ) = ( \begin{matrix}1 & 1 & … & 1 \\ {x}_{1} & {x}_{2} & … & {x}_{α} \\ {y}_{1} & {y}_{2} & … & {y}_{α} \\ {z}_{1} & {z}_{2} & … & {z}_{α} \\ {x}_{1} {y}_{1} & {x}_{2} {y}_{2} & … & {x}_{α} {y}_{α} \\ {y}_{1} {z}_{1} & {y}_{2} {z}_{2} & … & {y}_{α} {z}_{α} \\ {z}_{1} {x}_{1} & {z}_{2} {x}_{2} & … & {z}_{α} {x}_{α}\end{matrix} ) & ( 4 ) \\ a = {MP}^{- 1} & ( 5 ) \\ a = {MP}^{+} & ( 6 )\end{matrix}$$

In Equation (5), P⁻¹ is an inverse matrix of the third matrix P, and, in Equation (6), P⁺ is a pseudo-inverse matrix of the third matrix P.

According to this application example, if any position in a space where the first magnetic sensor and the second magnetic sensor are disposed is represented by a position vector r, and a first matrix of three rows and seven columns formed of the unknowns of Equation (1) is indicated by a, the magnetic field vector B of the second magnetic field at any position is expressed by Equation (2). A second matrix M of three rows and α columns expressed by Equation (2) is formed by using a measurement value (α detection magnetic field vectors Bₖ) in the second magnetic sensor. A third matrix P of seven rows and α columns expressed by Equation (4) is formed by using a position (α magnetic sensor term vectors Rₖ) of the second magnetic sensor. If the number α of second magnetic sensors is 7, an inverse matrix of the third matrix P is present, and thus the first matrix a is obtained by multiplying the second matrix M by the inverse matrix (P⁻¹) of the third matrix P as in Equation (5). If the number α of second magnetic sensors is 8 or larger, an inverse matrix of the third matrix P is not present, and thus the first matrix a is obtained by multiplying the second matrix M by a pseudo-inverse matrix (P⁺) of the third matrix P as in Equation (6). As mentioned above, the first matrix a corresponding to the unknowns of Equation (1) is obtained by using Equation (5) or (6), and thus it is possible to calculate an approximate value of the second magnetic field with high accuracy.

### Application Example 9

In the magnetic measurement system according to the application example, it is preferable that, when a first vector formed of unknowns of the above Equation (1) is indicated by b which is expressed by Equation (7), a second vector formed of the measurement value in the second magnetic sensor is indicated by N which is expressed by Equation (8), and a fourth matrix formed of a position of the second magnetic sensor is indicated by Q which is expressed by Equation (9), the first vector b is obtained by using Equation (10) or Equation (11).

$$\begin{matrix}\vec{b} ≡ ( \begin{matrix}{a}_{11} \\ {a}_{12} \\ ⋮ \\ {a}_{16} \\ {a}_{17} \\ {a}_{21} \\ ⋮ \\ {a}_{27} \\ {a}_{31} \\ ⋮ \\ {a}_{37}\end{matrix} ) = ( \begin{matrix}{b}_{1} \\ {b}_{2} \\ ⋮ \\ {b}_{6} \\ {b}_{7} \\ {b}_{8} \\ ⋮ \\ {b}_{14} \\ {b}_{15} \\ ⋮ \\ {b}_{21}\end{matrix} ) & ( 7 ) \\ \vec{N} ≡ ( \begin{matrix}{B}_{11} \\ {B}_{21} \\ {B}_{31} \\ {B}_{12} \\ {B}_{22} \\ {B}_{32} \\ ⋮ \\ {B}_{3 α - 1} \\ {B}_{1 α} \\ {B}_{2 α} \\ {B}_{3 α}\end{matrix} ) = ( \begin{matrix}{n}_{1} \\ {n}_{2} \\ {n}_{3} \\ {n}_{4} \\ {n}_{5} \\ {n}_{6} \\ ⋮ \\ {n}_{3 α - 3} \\ {n}_{3 α - 2} \\ {n}_{3 α - 1} \\ {n}_{3 α}\end{matrix} ) & ( 8 ) \\ \begin{matrix}Q ≡ ( \begin{matrix}{\vec{R}}_{1}^{T} & \vec{0} & \vec{0} \\ \vec{0} & {\vec{R}}_{1}^{T} & \vec{0} \\ \vec{0} & \vec{0} & {\vec{R}}_{1}^{T} \\ {\vec{R}}_{2}^{T} & \vec{0} & \vec{0} \\ \vec{0} & {\vec{R}}_{2}^{T} & \vec{0} \\ \vec{0} & \vec{0} & {\vec{R}}_{2}^{T} \\ \vec{0} & \vec{0} & {\vec{R}}_{α - 1}^{T} \\ {\vec{R}}_{α}^{T} & \vec{0} & \vec{0} \\ \vec{0} & {\vec{R}}_{α}^{T} & \vec{0} \\ \vec{0} & \vec{0} & {\vec{R}}_{α}^{T}\end{matrix} ) \\ = ( \begin{matrix}{R}_{11} & {R}_{21} & ⋯ & {R}_{61} & {R}_{71} & 0 & 0 & ⋯ & 0 & 0 & 0 & 0 & ⋯ & 0 & 0 \\ 0 & 0 & ⋯ & 0 & 0 & {R}_{11} & {R}_{21} & ⋯ & {R}_{61} & {R}_{71} & 0 & 0 & ⋯ & 0 & 0 \\ 0 & 0 & ⋯ & 0 & 0 & 0 & 0 & ⋯ & 0 & 0 & {R}_{11} & {R}_{21} & ⋯ & {R}_{61} & {R}_{71} \\ {R}_{12} & {R}_{22} & ⋯ & {R}_{62} & {R}_{72} & 0 & 0 & ⋯ & 0 & 0 & 0 & 0 & ⋯ & 0 & 0 \\ 0 & 0 & ⋯ & 0 & 0 & {R}_{12} & {R}_{22} & ⋯ & {R}_{62} & {R}_{72} & 0 & 0 & ⋯ & 0 & 0 \\ 0 & 0 & ⋯ & 0 & 0 & 0 & 0 & ⋯ & 0 & 0 & {R}_{12} & {R}_{22} & ⋯ & {R}_{62} & {R}_{72} \\ ⋮ & ⋮ & & ⋮ & ⋮ & ⋮ & ⋮ & & ⋮ & ⋮ & ⋮ & ⋮ & & ⋮ & ⋮ \\ 0 & 0 & ⋯ & 0 & 0 & 0 & 0 & ⋯ & 0 & 0 & {R}_{1 α - 1} & {R}_{2 α - 1} & ⋯ & {R}_{6 α - 1} & {R}_{7 α - 1} \\ {R}_{1 α} & {R}_{2 α} & ⋯ & {R}_{6 α} & {R}_{7 α} & 0 & 0 & ⋯ & 0 & 0 & 0 & 0 & ⋯ & 0 & 0 \\ 0 & 0 & ⋯ & 0 & 0 & {R}_{1 α} & {R}_{2 α} & ⋯ & {R}_{6 α} & {R}_{7 α} & 0 & 0 & ⋯ & 0 & 0 \\ 0 & 0 & ⋯ & 0 & 0 & 0 & 0 & ⋯ & 0 & 0 & {R}_{1 α} & {R}_{2 α} & ⋯ & {R}_{6 α} & {R}_{7 α}\end{matrix} )\end{matrix} & ( 9 ) \\ \vec{b} = {Q}^{- 1} \vec{N} & ( 10 ) \\ \vec{b} = {Q}^{+} \vec{N} & ( 11 )\end{matrix}$$

According to the configuration of this application example, the unknowns (3×7) of Equation (1) are arranged in one column, and thus a first vector b of twenty-one rows and one column expressed by Equation (7) is formed. A second vector N of (3×α) rows and one column expressed by Equation (8) is formed by using the measurement value (α detection magnetic field vectors Bₖ) in the second magnetic sensor. A fourth matrix Q expressed by Equation (9) is formed by using a position (α magnetic sensor term vectors Rₖ) of the second magnetic sensor. In Equation (9), a row vector Rₖ^{T} is a transposed matrix of the magnetic sensor term vector Rₖ and is a row vector of one row and seven columns, and a zero vector 0 is a row vector of one row and seven columns in which all matrix elements are zeros. If the number α of second magnetic sensors is 7, an inverse matrix of the fourth matrix Q is present, and thus the first vector b corresponding to the unknowns is obtained by multiplying the second vector N by an inverse matrix Q⁻¹ of the fourth matrix Q as in Equation (10). If the number α of second magnetic sensors is 8 or larger, an inverse matrix of the fourth matrix Q is not present, and thus the first vector b is obtained by multiplying the second vector N by a pseudo-inverse matrix (Q⁺) of the fourth matrix Q as in Equation (11). As mentioned above, the first vector b corresponding to the unknowns of Equation (1) is obtained by using Equation (10) or (11), and thus it is possible to calculate an approximate value of the second magnetic field with high accuracy.

### Application Example 10

In the magnetic measurement system according to the application example, it is preferable that the multi-variable polynomial is expressed by the above Equation (1) in consideration of Equation (12).

a₃₄=−(a₁₂+a₂₃)

a₃₇=−a₂₅

a₃₆=−a₁₅

a₂₆=−a₁₇ (12)

According to the configuration of this application example, four of the 21 unknowns are expressed by Equation (12) by applying Gauss' law regarding a magnetic field when solving Equation (1). Therefore, the four unknowns of the left side of Equation (12) are not required to be solved, and thus the number of unknowns to be obtained can be reduced to 17.

### Application Example 11

In the magnetic measurement system according to the application example, it is preferable that the non-linear polynomial is expressed by the above Equation (1) in consideration of the above Equation (12).

According to the configuration of this application example, four of the 21 unknowns are expressed by Equation (12) by applying Gauss' law regarding a magnetic field when solving Equation (1). Therefore, the four unknowns of the left side of Equation (12) are not required to be solved, and thus the number of unknowns to be obtained can be reduced to 17.

### Application Example 12

In the magnetic measurement system according to the application example, it is preferable that the second magnetic sensor measures 17 or more magnetic field vector components of the second magnetic field.

According to the configuration of this application example, since the number of unknowns to be obtained is 17, it is possible to compute an approximate value of the second magnetic field with high accuracy by measuring 17 or more magnetic field vector components of the second magnetic field.

### Application Example 13

In the magnetic measurement system according to the application example, it is preferable that, when a third vector formed of unknowns of the above Equation (1) is indicated by c which is expressed by Equation (13), a second vector formed of the measurement value in the second magnetic sensor is indicated by N which is expressed by the above Equation (8), and a fifth matrix formed of a position of the second magnetic sensor is indicated by S which is expressed by Equations (14) and (15), or Equation (16), the third vector c is obtained by using Equation (17).

$$\begin{matrix}\vec{c} ≡ ( \begin{matrix}{a}_{11} \\ {a}_{12} \\ {a}_{13} \\ {a}_{14} \\ {a}_{15} \\ {a}_{16} \\ {a}_{17} \\ {a}_{21} \\ {a}_{22} \\ {a}_{23} \\ {a}_{24} \\ {a}_{25} \\ {a}_{27} \\ {a}_{31} \\ {a}_{32} \\ {a}_{33} \\ {a}_{35}\end{matrix} ) & ( 13 ) \\ S = ( \begin{matrix}{T}_{1} \\ {T}_{2} \\ {T}_{3} \\ ⋮ \\ {T}_{α}\end{matrix} ) & ( 14 ) \\ \begin{matrix}{T}_{k} = ( \begin{matrix}{R}_{1 k} & {R}_{2 k} & {R}_{3 k} & {R}_{4 k} & {R}_{5 k} & {R}_{6 k} & {R}_{7 k} & 0 & 0 & 0 & 0 & 0 & 0 & 0 & 0 & 0 & 0 \\ 0 & 0 & 0 & 0 & 0 & 0 & - {R}_{6 k} & {R}_{1 k} & {R}_{2 k} & {R}_{3 k} & {R}_{4 k} & {R}_{5 k} & {R}_{7 k} & 0 & 0 & 0 & 0 \\ 0 & - {R}_{2 k} & 0 & 0 & - {R}_{6 k} & 0 & 0 & 0 & 0 & - {R}_{4 k} & 0 & - {R}_{7 k} & 0 & {R}_{1 k} & {R}_{2 k} & {R}_{3 k} & {R}_{4 k}\end{matrix} ) \\ = ( \begin{matrix}1 & {x}_{k} & {y}_{k} & {z}_{k} & {x}_{k} {y}_{k} & {y}_{k} {z}_{k} & {z}_{k} {x}_{k} & 0 & 0 & 0 & 0 & 0 & 0 & 0 & 0 & 0 & 0 \\ 0 & 0 & 0 & 0 & 0 & 0 & - {y}_{k} {z}_{k} & 1 & {x}_{k} & {y}_{k} & {z}_{k} & {x}_{k} {y}_{k} & {z}_{k} {x}_{k} & 0 & 0 & 0 & 0 \\ 0 & - {z}_{k} & 0 & 0 & - {y}_{k} {z}_{k} & 0 & 0 & 0 & 0 & - {z}_{k} & 0 & - {z}_{k} {x}_{k} & 0 & 1 & {x}_{k} & {y}_{k} & {x}_{k} {y}_{k}\end{matrix} )\end{matrix} & ( 15 ) \\ S ≡ ( \begin{matrix}{R}_{11} & {R}_{21} & {R}_{31} & {R}_{41} & {R}_{51} & {R}_{61} & {R}_{71} & 0 & 0 & 0 & 0 & 0 & 0 & 0 & 0 & 0 & 0 \\ 0 & 0 & 0 & 0 & 0 & 0 & - {R}_{61} & {R}_{11} & {R}_{21} & {R}_{31} & {R}_{41} & {R}_{51} & {R}_{71} & 0 & 0 & 0 & 0 \\ 0 & - {R}_{41} & 0 & 0 & - {R}_{61} & 0 & 0 & 0 & 0 & - {R}_{41} & 0 & - {R}_{71} & 0 & {R}_{11} & {R}_{21} & {R}_{31} & {R}_{51} \\ {R}_{12} & {R}_{22} & {R}_{32} & {R}_{42} & {R}_{52} & {R}_{62} & {R}_{72} & 0 & 0 & 0 & 0 & 0 & 0 & 0 & 0 & 0 & 0 \\ 0 & 0 & 0 & 0 & 0 & 0 & - {R}_{62} & {R}_{12} & {R}_{22} & {R}_{32} & {R}_{42} & {R}_{52} & {R}_{72} & 0 & 0 & 0 & 0 \\ 0 & - {R}_{42} & 0 & 0 & - {R}_{62} & 0 & 0 & 0 & 0 & - {R}_{42} & 0 & - {R}_{32} & 0 & {R}_{12} & {R}_{22} & {R}_{32} & {R}_{52} \\ {R}_{13} & {R}_{23} & {R}_{33} & {R}_{43} & {R}_{53} & {R}_{63} & {R}_{73} & 0 & 0 & 0 & 0 & 0 & 0 & 0 & 0 & 0 & 0 \\ 0 & 0 & 0 & 0 & 0 & 0 & - {R}_{63} & {R}_{13} & {R}_{23} & {R}_{33} & {R}_{43} & {R}_{53} & {R}_{73} & 0 & 0 & 0 & 0 \\ 0 & - {R}_{43} & 0 & 0 & - {R}_{63} & 0 & 0 & 0 & 0 & - {R}_{43} & 0 & - {R}_{73} & 0 & {R}_{13} & {R}_{23} & {R}_{33} & {R}_{53} \\ {R}_{14} & {R}_{24} & {R}_{34} & {R}_{44} & {R}_{54} & {R}_{64} & {R}_{74} & 0 & 0 & 0 & 0 & 0 & 0 & 0 & 0 & 0 & 0 \\ 0 & 0 & 0 & 0 & 0 & 0 & - {R}_{64} & {R}_{14} & {R}_{24} & {R}_{34} & {R}_{44} & {R}_{54} & {R}_{74} & 0 & 0 & 0 & 0 \\ 0 & - {R}_{44} & 0 & 0 & - {R}_{64} & 0 & 0 & 0 & 0 & - {R}_{44} & 0 & - {R}_{74} & 0 & {R}_{14} & {R}_{24} & {R}_{34} & {R}_{54} \\ ⋮ & & & & & & & & & & & & & & & & ⋮ \\ ⋮ & & & & & & & & & & & & & & & & ⋮ \\ ⋮ & & & & & & & & & & & & & & & & ⋮ \\ {R}_{1 k} & {R}_{2 k} & {R}_{3 k} & {R}_{4 k} & {R}_{5 k} & {R}_{6 k} & {R}_{7 k} & 0 & 0 & 0 & 0 & 0 & 0 & 0 & 0 & 0 & 0 \\ 0 & 0 & 0 & 0 & 0 & 0 & - {R}_{6 k} & {R}_{1 k} & {R}_{2 k} & {R}_{3 k} & {R}_{4 k} & {R}_{5 k} & {R}_{7 k} & 0 & 0 & 0 & 0 \\ 0 & - {R}_{4 k} & 0 & 0 & - {R}_{6 k} & 0 & 0 & 0 & 0 & - {R}_{4 k} & 0 & - {R}_{7 k} & 0 & {R}_{1 k} & {R}_{2 k} & {R}_{3 k} & {R}_{5 k} \\ ⋮ & & & & & & & & & & & & & & & & ⋮ \\ ⋮ & & & & & & & & & & & & & & & & ⋮ \\ ⋮ & & & & & & & & & & & & & & & & ⋮ \\ {R}_{1 α} & {R}_{2 α} & {R}_{3 α} & {R}_{4 α} & {R}_{5 α} & {R}_{6 α} & {R}_{7 α} & 0 & 0 & 0 & 0 & 0 & 0 & 0 & 0 & 0 & 0 \\ 0 & 0 & 0 & 0 & 0 & 0 & - {R}_{6 α} & {R}_{1 α} & {R}_{2 α} & {R}_{3 α} & {R}_{4 α} & {R}_{5 α} & {R}_{7 α} & 0 & 0 & 0 & 0 \\ 0 & - {R}_{4 α} & 0 & 0 & - {R}_{6 α} & 0 & 0 & 0 & 0 & - {R}_{4 α} & 0 & - {R}_{7 α} & 0 & {R}_{1 α} & {R}_{2 α} & {R}_{3 α} & {R}_{5 α}\end{matrix} ) & ( 16 ) \\ \vec{c} = {S}^{+} \vec{N} & ( 17 )\end{matrix}$$

According to the configuration of this application example, the 17 unknowns are arranged in one column except for the 4 unknowns of the left side of Equation (12) among the 21 unknowns, and thus a third vector c of seventeen rows and one column expressed by Equation (13) is formed. A fifth matrix S of 3α rows and seventeen columns expressed by Equation (16) is formed by using a position (a magnetic sensor term vectors Rₖ) of the second magnetic sensor. Here, in Equation (16), a single magnetic sensor term vector Rₖ corresponds to three rows of 3α rows, and, if a (3k−2)-th row, a (3k−1)-th row, and a 3k-th row are arranged in a partial matrix Tₖ of three rows and seventeen columns, the partial matrix Tₖ is expressed by Equation (15). In a case of using the partial matrix Tₖ, the fifth matrix S is a matrix obtained by arranging a partial matrices including the partial matrix T₁ of k=1 to the partial matrix T_{α} of k=α in α rows and one column, and is expressed by Equation (14). If the number α of second magnetic sensors is 6 or larger, the number of rows of the second vector N expressed by Equation (8) is 18 or larger in relation to the number 17 of unknowns, and thus the 17 unknowns can be specified by using a least square method. In this case, an inverse matrix of the fifth matrix S is not present, and thus the third vector c corresponding to the unknowns is obtained by multiplying the second vector N by a pseudo-inverse matrix (S⁺) of the fifth matrix S as in Equation (17).

### Application Example 14

A magnetic measurement system according to this application example includes a first magnetic sensor that measures a first magnetic field and a second magnetic field; a first-second magnetic sensor and a second-second magnetic sensor that are disposed around the first magnetic sensor; and a processing apparatus that computes an approximate value of the second magnetic field in the first magnetic sensor by using a measurement value in the first-second magnetic sensor and a measurement value in the second-second magnetic sensor, in which the first magnetic sensor is disposed at a position including the centroid between the first-second magnetic sensor and the second-second magnetic sensor.

According to the configuration of this application example, the first magnetic sensor measuring the first magnetic field and the second magnetic field is disposed at a position including the centroid between the first-second magnetic sensor and the second-second magnetic sensor. In other words, the first-second magnetic sensor and the second-second magnetic sensor are disposed so as to be symmetric to each other with respect to the first magnetic sensor. For this reason, the processing apparatus uses a measurement value in the first-second magnetic sensor and the second-second magnetic sensor with the same importance, and thus it is possible to compute an approximate value of the second magnetic field in the first magnetic sensor with high accuracy.

### Application Example 15

It is preferable that the magnetic measurement system according to the application example further includes a third-second magnetic sensor and a fourth-second magnetic sensor, and the third-second magnetic sensor and the fourth-second magnetic sensor are disposed at positions which are symmetric to each other with respect to the centroid, and a line segment connecting the first-second magnetic sensor to the second-second magnetic sensor intersects a line segment connecting the third-second magnetic sensor to the fourth-second magnetic sensor.

According to the configuration of this application example, the third-second magnetic sensor and the fourth-second magnetic sensor are disposed at positions which are symmetric to each other with respect to the centroid between the first-second magnetic sensor and the second-second magnetic sensor, and are thus disposed to be symmetric to each other with respect to the first magnetic sensor. Thus, the processing apparatus uses a measurement value in the third-second magnetic sensor and the fourth-second magnetic sensor with the same importance, and thus it is possible to compute an approximate value of the second magnetic field in the first magnetic sensor with high accuracy. Since the first-second magnetic sensor, the second-second magnetic sensor, the third-second magnetic sensor, and the fourth-second magnetic sensor are not disposed in a straight line and are disposed planarly (in a two-dimensional manner), it is possible to compute an approximate value of the second magnetic field in the first magnetic sensor in a two-dimensional manner with high accuracy.

### Application Example 16

A magnetic measurement system according to this application example includes a first magnetic sensor that measures a first magnetic field and a second magnetic field; a first-second magnetic sensor, a second-second magnetic sensor, a third-second magnetic sensor, and a fourth-second magnetic sensor that are disposed around the first magnetic sensor; and a processing apparatus that computes an approximate value of the second magnetic field in the first magnetic sensor by using a measurement value in the first-second magnetic sensor, a measurement value in the second-second magnetic sensor, a measurement value in the third-second magnetic sensor, and a measurement value in the fourth-second magnetic sensor, in which the first magnetic sensor is disposed at a position including an intersection portion at which a line segment connecting the first-second magnetic sensor to the second-second magnetic sensor intersects a line segment connecting the third-second magnetic sensor to the fourth-second magnetic sensor.

According to the configuration of this application example, the first magnetic sensor measuring the first magnetic field and the second magnetic field is disposed at a position including an intersection portion at which a line segment connecting the first-second magnetic sensor to the second-second magnetic sensor intersects a line segment connecting the third-second magnetic sensor to the fourth-second magnetic sensor. The first-second magnetic sensor, the second-second magnetic sensor, the third-second magnetic sensor, and the fourth-second magnetic sensor are not disposed in a straight line and are disposed planarly (in a two-dimensional manner). Therefore, the processing apparatus can compute an approximate value of the second magnetic field in the first magnetic sensor in a two-dimensional manner with high accuracy.

### Application Example 17

In the magnetic measurement system according to the application example, it is preferable that a line segment connecting the first-second magnetic sensor to the second-second magnetic sensor is orthogonal to a line segment connecting the third-second magnetic sensor to the fourth-second magnetic sensor.

According to the configuration of this application example, an approximate value of the second magnetic field in the first magnetic sensor in a direction along the line segment connecting the first-second magnetic sensor to the second-second magnetic sensor can be computed with high accuracy by using a measurement value in the first-second magnetic sensor and the second-second magnetic sensor. An approximate value of the second magnetic field in the first magnetic sensor in a direction along the line segment connecting the third-second magnetic sensor to the fourth-second magnetic sensor can be computed with high accuracy by using a measurement value in the third-second magnetic sensor and the fourth-second magnetic sensor. Since the line segment connecting the first-second magnetic sensor to the second-second magnetic sensor is orthogonal to the line segment connecting the third-second magnetic sensor to the fourth-second magnetic sensor, the processing apparatus can compute an approximate value of the second magnetic field in the first magnetic sensor in a two-dimensional manner with higher accuracy.

### BRIEF DESCRIPTION OF THE DRAWINGS

The invention will be described with reference to the accompanying drawings, wherein like numbers reference like elements.

FIG. 1 is a schematic side view illustrating an example of a configuration of a magnetic measurement system according to a first embodiment.

FIGS. 2A and 2B are schematic diagrams illustrating a structure of a heart magnetic field sensor according to the first embodiment.

FIG. 3 is a flowchart illustrating a schematic heart magnetic field measurement process according to the first embodiment.

FIGS. 4A and 4B are diagrams illustrating arrangement of noise magnetic sensors related to Example 1-1.

FIGS. 5A and 5B are diagrams illustrating arrangement of noise magnetic sensors related to Example 1-1.

FIGS. 6A and 6B are diagrams illustrating arrangement of noise magnetic sensors related to Example 1-3.

FIGS. 7A and 7B are diagrams illustrating arrangement of noise magnetic sensors related to Example 1-3.

FIGS. 8A and 8B are diagrams illustrating arrangement of noise magnetic sensors related to Example 3-1.

FIGS. 9A and 9B are diagrams illustrating arrangement of noise magnetic sensors related to Example 3-1.

### DESCRIPTION OF EXEMPLARY EMBODIMENTS

Hereinafter, embodiments will be described with reference to the drawings.

In addition, respective members in the drawings are illustrated in different scales in order to be recognizable in the drawings.

### First Embodiment

### Magnetic Measurement System

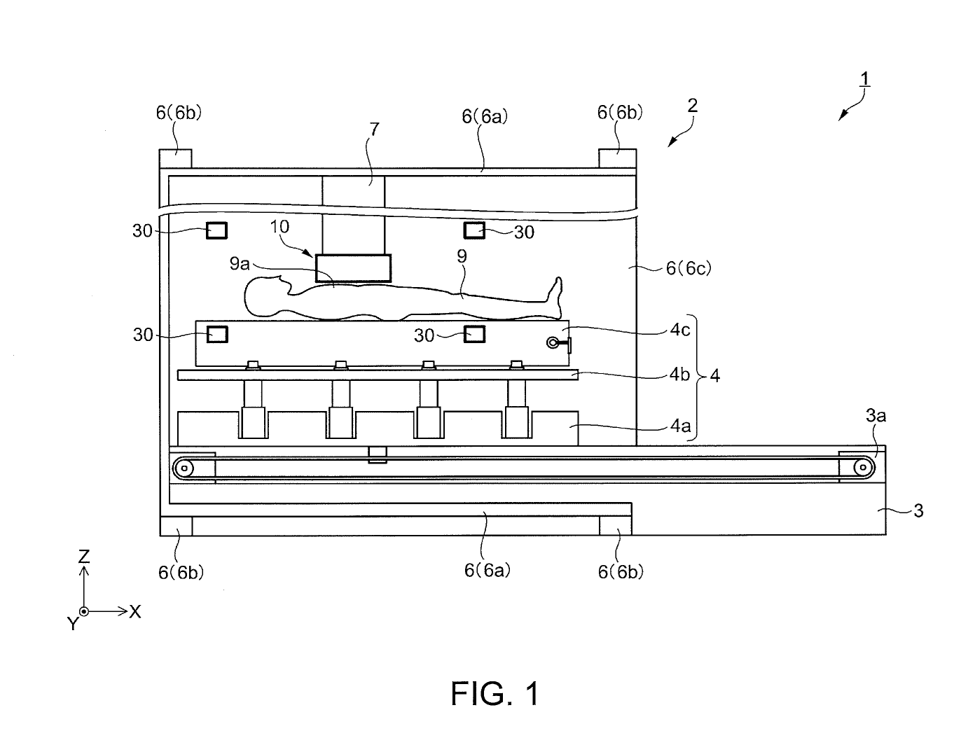

First, a configuration example of a magnetic measurement system according to a first embodiment will be described. FIG. 1 is a schematic side view illustrating an example of a configuration of a magnetic measurement system according to the first embodiment. A magnetic measurement system 1 illustrated in FIG. 1 is a system measuring a heart magnetic field generated from the heart of a subject (living body) 9 as a measurement target object. As illustrated in FIG. 1, the magnetic measurement system 1 includes a heart magnetic field sensor 10 as a first magnetic sensor, noise magnetic sensors 30 as a second magnetic sensor, and a magnetic measurement apparatus 2 as a processing apparatus.

The heart magnetic field sensor 10 is a sensor measuring a weak first magnetic field such as a heart magnetic field or a brain magnetic field, and a second magnetic field such as an external magnetic field (magnetic noise), and is used as a magnetoencephalograph, a magnetocardiograph, or the like. Each of the noise magnetic sensors 30 is a sensor measuring the second magnetic field such as an external magnetic field (magnetic noise). As the heart magnetic field sensor 10 and the noise magnetic sensors 30, an optical pumping magnetic sensor, a SQUID type magnetic sensor, a flux gate magnetic sensor, an MI sensor, a hole element, and the like may be used.

The magnetic measurement apparatus 2 includes a foundation 3, a table 4, and a magnetic shield device 6. A height direction (upper-and-lower direction in FIG. 1) of the magnetic measurement apparatus 2 is set to a Z direction. The Z direction is a vertical direction. Directions in which upper surfaces of the foundation 3 and the table 4 extend are set to an X direction and a Y direction. The X direction and the Y direction are a horizontal direction, and the X direction and the Y direction are orthogonal to each other. A height direction (leftward-and-rightward direction in FIG. 1) of a subject 9 who is lying down is set to the X direction.

The foundation 3 is disposed on a bottom surface inside the magnetic shield device 6 (main body 6a) and extends to the outside of the main body 6a in the X direction (direction in which the subject 9 can be moved). The table 4 includes an X-direction table 4a, a Z-direction table 4b, and a Y-direction table 4c. The X-direction table 4a which is moved in the X direction by an X-direction linear motion mechanism 3a is provided on the foundation 3. The Z-direction table 4b which is lifted in the Z direction by a lifting device (not illustrated) is provided on the X-direction table 4a. The Y-direction table 4c which is moved on a rail in the Y direction by a Y-direction linear motion mechanism (not illustrated) is provided on the Z-direction table 4b.

The magnetic shield device 6 includes a rectangular tubular main body 6a having an opening 6c. The inside of the main body 6a is hollow, and a sectional shape of surfaces (orthogonal planes in the X direction in the Y-Z section) passing through the Y direction and the Z direction is a substantially quadrangle shape. When a heart magnetic field is measured, the subject 9 is accommodated inside the main body 6a in a lying state on the table 4. The main body 6a extends in the X direction, and thus functions as a passive magnetic shield.

The heart magnetic field sensor 10 and the noise magnetic sensors 30 are disposed inside the main body 6a of the magnetic shield device 6. The magnetic shield device 6 prevents an external magnetic field such as terrestrial magnetism from entering a space where the heart magnetic field sensor 10 is disposed. In other words, a magnetic field in the space where the heart magnetic field sensor 10 is disposed is considerably lower than the external magnetic field, and thus the influence of the external magnetic field on the heart magnetic field sensor 10 is minimized by the magnetic shield device 6.

The foundation 3 protrudes out of the opening 6c of the main body 6a in the +X direction. As a size of the magnetic shield device 6, a length thereof in the X direction is about 200 cm, for example, and one side of the opening 6c is about 90 cm. The subject 9 laid down on the table 4 can be moved so as to come in and out of the magnetic shield device 6 through the opening 6c along with the table 4 in the X direction on the foundation 3.

Although not illustrated, the magnetic measurement apparatus 2 includes a controller controlling the magnetic measurement apparatus 2 by using an electric signal. A magnetic field or a residual magnetic field is generated due to the electric signal, and thus becomes noise when detected by the heart magnetic field sensor 10. The controller is provided at a location separated from the opening 6c of the magnetic shield device 6 so that the generated magnetic field or residual magnetic field hardly reaches the heart magnetic field sensor 10.

The controller is provided with a display device and an input device. The display device is an LCD or an OLED, and displays a measurement situation, a measurement result, and the like. The input device is constituted of a keyboard, a rotary knob, or the like. An operator operates the input device so as to input various instructions such as a measurement starting instruction or a measurement condition to the magnetic measurement apparatus 2.

The main body 6a of the magnetic shield device 6 is made of a ferromagnetic material having relative permeability of for example, several thousand or more, or a conductor having high conductivity. As the ferromagnetic material, permalloy, ferrite, iron, chromium, cobalt-based amorphous metal, or the like may be used. As the conductor having high conductivity, for example, aluminum which has a magnetic field reduction function due to an eddy current effect may be used. The main body 6a may be formed by alternately stacking a ferromagnetic material and a conductor having high conductivity.

Correction coils (Helmholtz coils) 6b are provided at ends on the +X direction side and −X direction side of the main body 6a and the foundation 3. A shape of each of the correction coils 6b is a frame shape, and the correction coil 6b is disposed to surround the main body 6a. The correction coil 6b is a coil for correcting an entering magnetic field which enters the internal space of the main body 6a. The entering magnetic field indicates an external magnetic field which passes through the opening 6c and enters the internal space. The entering magnetic field is strongest in the X direction with respect to the opening 6c. The correction coil 6b generates a magnetic field which cancels out the entering magnetic field by using a current supplied from the controller.

The heart magnetic field sensor 10 is fixed to a ceiling of the main body 6a via a support member 7. The heart magnetic field sensor 10 measures a strength component of a magnetic field in the Z direction. When a heart magnetic field of the subject 9 is measured, the X-direction table 4a and the Y-direction table 4c are moved so that, in the subject 9, a chest 9a as a measurement position faces the heart magnetic field sensor 10, and the Z-direction table 4b is moved up so that the chest 9a comes close to the heart magnetic field sensor 10.

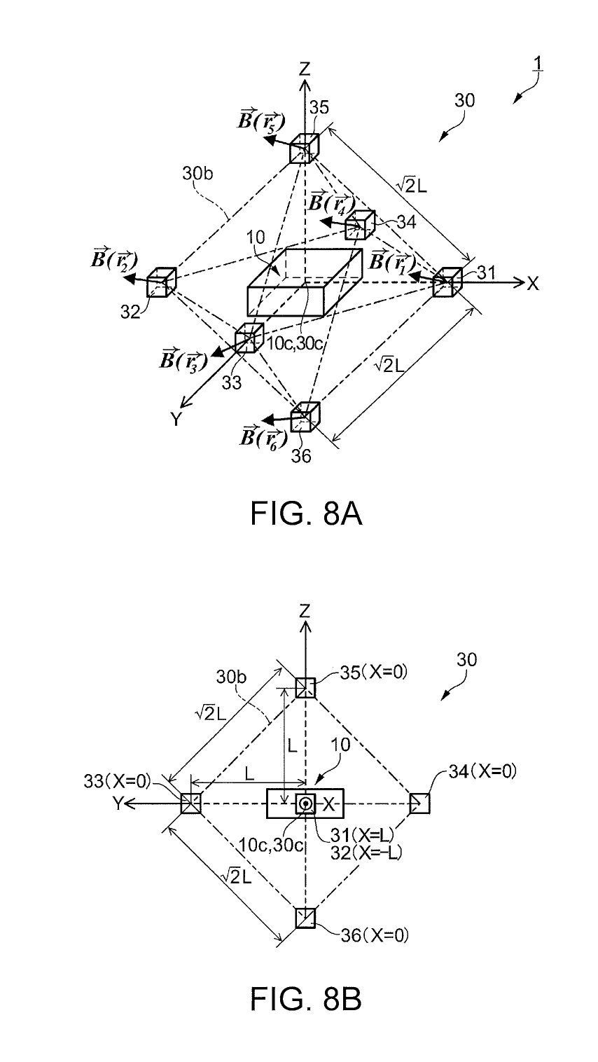

A plurality of (six or more) noise magnetic sensors 30 are disposed around the heart magnetic field sensor 10. As will be described later in detail, the magnetic measurement system 1 includes, as the noise magnetic sensors 30, a first noise magnetic sensor 31, a second noise magnetic sensor 32, a third noise magnetic sensor 33, a fourth noise magnetic sensor 34, a fifth noise magnetic sensor 35, and a sixth noise magnetic sensor 36 (refer to FIG. 8A).

Each of the noise magnetic sensors 30 (31, 32, 33, 34, 35, and 36) measures three components of a magnetic field in the X direction, the Y direction, and the Z direction. Consequently, it is possible to specify a magnetic field distribution around the noise magnetic sensors 30. The noise magnetic sensors 30 are preferably disposed in a stereoscopic manner so as to surround a space (hereinafter, referred to as a measurement target space) in which a magnetic field distribution is desired to be specified, that is, the space in which the subject 9 is disposed.

The controller of the magnetic measurement apparatus 2 has a function of calculating a heart magnetic field of the subject 9 which is the first magnetic field on the basis of measurement values of the first magnetic field and a measurement value of the second magnetic field measured by the heart magnetic field sensor 10, and measurement values of the second magnetic field measured by the noise magnetic sensors 30. More specifically, the controller computes an approximate value of the second magnetic field in the heart magnetic field sensor 10 by using measurement values in the noise magnetic sensors 30 (31, 32, 33, 34, 35, and 36), and calculates the first magnetic field by subtracting the approximate value from a value measured by in the heart magnetic field sensor 10.

The heart magnetic field sensor 10 is preferably disposed at a position including an intersection portion at which a line segment connecting the first noise magnetic sensor 31 to the second noise magnetic sensor 32, a line segment connecting the third noise magnetic sensor 33 to the fourth noise magnetic sensor 34, and a line segment connecting the fifth noise magnetic sensor 35 to the sixth noise magnetic sensor 36, intersect each other.

As mentioned above, in a case where the number of noise magnetic sensors 30 is 2n or 2n+1 (where n is an integer of 3 or greater), and n pairs of noise magnetic sensors 30 are set, it is preferable from the viewpoint of highly accurate measurement that the heart magnetic field sensor 10 is disposed at a position including the centroid of the two noise magnetic sensors 30 for each pair. In other words, preferably, two noise magnetic sensors 30 of each pair are disposed symmetrically with respect to the heart magnetic field sensor 10.

The heart magnetic field sensor 10 is preferably disposed so that, among the line segment connecting the first noise magnetic sensor 31 to the second noise magnetic sensor 32, the line segment connecting the third noise magnetic sensor 33 to the fourth noise magnetic sensor 34, and the line segment connecting the fifth noise magnetic sensor 35 to the sixth noise magnetic sensor 36, at least two line segments are orthogonal to each other, and the one remaining line segment intersects a plane which is parallel to the two line segments. To summarize, the noise magnetic sensors 30 including the first noise magnetic sensor 31 to the sixth noise magnetic sensor 36 are preferably disposed in a stereoscopic manner. Details of arrangement of the noise magnetic sensors 30 will be described later.

### Heart Magnetic Field Sensor

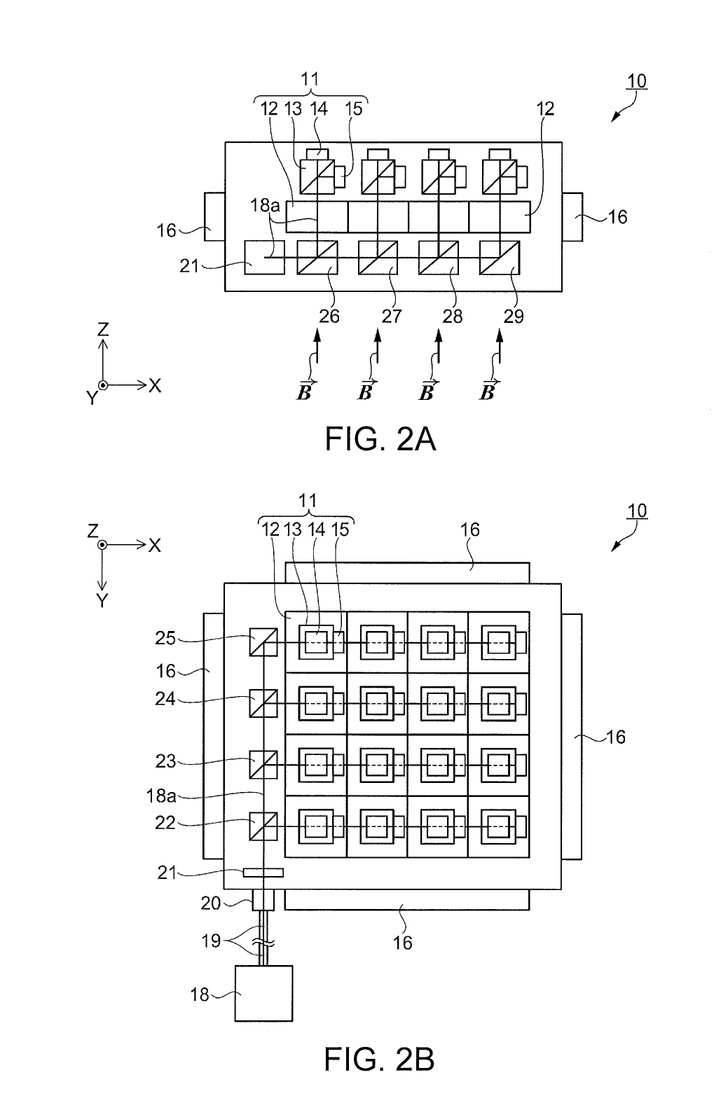

Next, a schematic structure of the heart magnetic field sensor 10 will be described. FIGS. 2A and 2B are schematic diagrams illustrating a structure of the heart magnetic field sensor according to the first embodiment. Specifically, FIG. 2A is a schematic side view of the heart magnetic field sensor, and FIG. 2B is a schematic plan view of the heart magnetic field sensor.

As illustrated in FIG. 2B, laser light 18a is supplied from a laser light source 18 to the heart magnetic field sensor 10. The laser light source 18 is provided in the controller, and the laser light 18a emitted from the laser light source 18 is supplied to the heart magnetic field sensor 10 through an optical fiber 19. The heart magnetic field sensor 10 is coupled to the optical fiber 19 via an optical connector 20.

The laser light source 18 outputs the laser light 18a with a wavelength corresponding to an absorption line of cesium. A wavelength of the laser light 18a is not particularly limited, but is set to a wavelength of 894 nm corresponding to the Dl-line, in the present embodiment. The laser light source 18 is a tunable laser device, and the laser light 18a output from the laser light source 18 is continuous light with a predetermined light amount.

The laser light 18a supplied via the optical connector 20 travels in the −Y direction and is incident to a polarization plate 21. The laser light 18a having passed through the polarization plate 21 is linearly polarized. The laser light 18a is sequentially incident to a first half mirror 22, a second half mirror 23, a third half mirror 24, and a first reflection mirror 25.

Some of the laser light 18a is reflected by the first half mirror 22, the second half mirror 23, and the third half mirror 24 so as to travel in the +X direction, and the other light is transmitted therethrough so as to travel in the −Y direction. The first reflection mirror 25 reflects the entire incident laser light 18a in the +X direction. An optical path of the laser light 18a is divided into four optical paths by the first half mirror 22, the second half mirror 23, the third half mirror 24, and the first reflection mirror 25. Reflectance of each mirror is set so that light intensities of the laser light beams 18a on the respective optical paths are the same as each other.

Next, as illustrated in FIG. 2A, the laser light 18a is sequentially applied and incident to a fourth half mirror 26, a fifth half mirror 27, a sixth half mirror 28, and a second reflection mirror 29. Some of the laser light 18a is reflected by the fourth half mirror 26, the fifth half mirror 27, and the sixth half mirror 28 so as to travel in the +Z direction, and the other light is transmitted therethrough so as to travel in the +X direction. The second reflection mirror 29 reflects the entire incident laser light 18a in the +Z direction.

A single optical path of the laser light 18a is divided into four optical paths by the fourth half mirror 26, the fifth half mirror 27, the sixth half mirror 28, and the second reflection mirror 29. Reflectance of each mirror is set so that light intensities of the laser light beams 18a on the respective optical paths are the same as each other. Therefore, the optical path of the laser light 18a is divided into the sixteen optical paths. In addition, reflectance of each mirror is set so that light intensities of the laser light beams 18a on the respective optical paths are the same as each other.

The sixteen gas cells 12 of four rows and four columns are provided on the respective optical paths of the laser light 18a on the +Z direction side of the fourth half mirror 26, the fifth half mirror 27, the sixth half mirror 28, and the second reflection mirror 29. The laser light beams 18a reflected by the fourth half mirror 26, the fifth half mirror 27, the sixth half mirror 28, and the second reflection mirror 29 pass through the gas cells 12. The gas cell 12 is a box having a cavity therein, and an alkali metal gas is enclosed in the cavity. The alkali metal is not particularly limited, and potassium, rubidium, or cesium may be used. In the present embodiment, for example, cesium is used as the alkali metal.

A polarization separator 13 is provided on the +Z direction side of each gas cell 12. The polarization separator 13 is an element which separates the incident laser light 18a into two polarization components of the laser light 18a, which are orthogonal to each other. As the polarization separator 13, for example, a Wollaston prism or a polarized beam splitter may be used.

A first photodetector 14 is provided on the +Z direction side of the polarization separator 13, and a second photodetector 15 is provided on the +X direction side of the polarization separator 13. The laser light 18a having passed through the polarization separator 13 is incident to the first photodetector 14, and the laser light 18a reflected by the polarization separator 13 is incident to the second photodetector 15. The first photodetector 14 and the second photodetector 15 output currents corresponding to an amount of incident laser light 18a to the controller.

If the first photodetector 14 and the second photodetector 15 generate magnetic fields, this may influence measurement, and thus the first photodetector 14 and the second photodetector 15 are preferably made of a non-magnetic material. The heart magnetic field sensor 10 includes heaters 16 which are provided on both sides in the X direction and both sides in the Y direction. Each of the heaters 16 preferably has a structure in which a magnetic field is not generated, and may employ, for example, a heater of a type of performing heating by causing steam or hot air to pass through a flow passage. Instead of using a heater, the gas cell 12 may be inductively heated by using a high frequency voltage.

The heart magnetic field sensor 10 is disposed on the +Z direction side of the subject 9 (refer to FIG. 1). A magnetic vector B generated from the subject 9 enters the heart magnetic field sensor 10 from the −Z direction side. The magnetic vector B passes through the fourth half mirror 26 to the second reflection mirror 29, successively passes through the gas cell 12, then passes through the polarization separator 13, and comes out of the heart magnetic field sensor 10.

The heart magnetic field sensor 10 is a sensor which is called an optical pumping type magnetic sensor or an optical pumping atom magnetic sensor. Cesium in the gas cell 12 is heated and is brought into a gaseous state. The cesium gas is irradiated with the linearly polarized laser light 18a, and thus cesium atoms are excited. Therefore, orientations of magnetic moments can be aligned. When the magnetic vector B passes through the gas cell 12 in this state, the magnetic moments of the cesium atoms precess due to a magnetic field of the magnetic vector B. This precession is referred to as Larmore precession.

The magnitude of the Larmore precession has a positive correlation with the strength of the magnetic vector B. In the Larmore precession, a polarization plane of the laser light 18a is rotated. The magnitude of the Larmore precession has a positive correlation with a change amount of a rotation angle of the polarization plane of the laser light 18a. Therefore, the strength of the magnetic vector B has a positive correlation with the change amount of a rotation angle of the polarization plane of the laser light 18a. The sensitivity of the heart magnetic field sensor 10 is high in the Z direction in the magnetic vector B, and is low in the direction orthogonal to the Z direction.

The polarization separator 13 separates the laser light 18a into two components of linearly polarized light which are orthogonal to each other. The first photodetector 14 and the second photodetector 15 detect the strengths of the two orthogonal components of linearly polarized light. Thus, the first photodetector 14 and the second photodetector 15 can detect a rotation angle of a polarization plane of the laser light 18a. The heart magnetic field sensor 10 can detect the strength of the magnetic vector B on the basis of a change of the rotation angle of the polarization plane of the laser light 18a.

An element constituted of the gas cell 12, the polarization separator 13, the first photodetector 14, and the second photodetector 15 is referred to as a sensor element 11. In the present embodiment, sixteen sensor elements 11 of four rows and four columns are disposed in the heart magnetic field sensor 10. The number and an arrangement of the sensor elements 11 in the heart magnetic field sensor 10 are not particularly limited. The sensor elements 11 may be disposed in three or less rows or five or more rows. Similarly, the sensor elements 11 may be disposed in three or less columns or five or more columns. The larger the number of sensor elements 11, the higher the spatial resolution.

An external magnetic field is prevented from entering the measurement target space in which the heart magnetic field sensor 10 is disposed by the magnetic shield device 6 (refer to FIG. 1), but it is difficult to completely prevent an external magnetic field from entering the measurement target space. In other words, a heart magnetic field and an external magnetic field (magnetic noise) are applied to the heart magnetic field sensor 10. For this reason, a measurement value obtained through measurement in the heart magnetic field sensor 10 includes a signal component based on the heart magnetic field and a noise component based on the external magnetic field. Therefore, in order to acquire an accurate heart magnetic field of the subject 9, it is necessary to remove the noise component from the measurement value obtained by the heart magnetic field sensor 10, with high accuracy.

### Noise Magnetic Sensor

Referring to FIG. 1 again, the noise magnetic sensors 30 are used to measure an external magnetic field (magnetic noise) in the measurement target space in which the heart magnetic field sensor 10 is disposed. An external magnetic field in the measurement target space is specified by using measurement values obtained by the noise magnetic sensors 30, and thus the external magnetic field (magnetic noise) can be removed from a measurement value obtained by the heart magnetic field sensor 10. It is assumed that the noise magnetic sensor 30 detects the second magnetic field such as the external magnetic field (magnetic noise) and does not detect the first magnetic field. If the noise magnetic sensor 30 has high sensitivity, a combined magnetic field of the first magnetic field and the second magnetic field may be measured by using a measurement value in the noise magnetic sensor 30.

The type of sensor used as the noise magnetic sensor 30 is not limited, but, for example, the same optical pumping type magnetic sensor as the heart magnetic field sensor 10 may be used. In a case of using the optical pumping type magnetic sensor, for example, three sensor elements 11 illustrated in FIGS. 2A and 2B may be combined and be used as a single noise magnetic sensor 30. In this case, the three sensor elements 11 measure magnetic vectors in respective directions including the X direction, the Y direction, and the Z direction. A single sensor element 11 may be used as a single noise magnetic sensor 30, irradiation may be performed with the laser light 18a in the respective directions including the X direction, the Y direction, and the Z direction, and magnetic vectors in the respective directions may be measured in a time series.

### Schematic Heart Magnetic Field Measurement Process

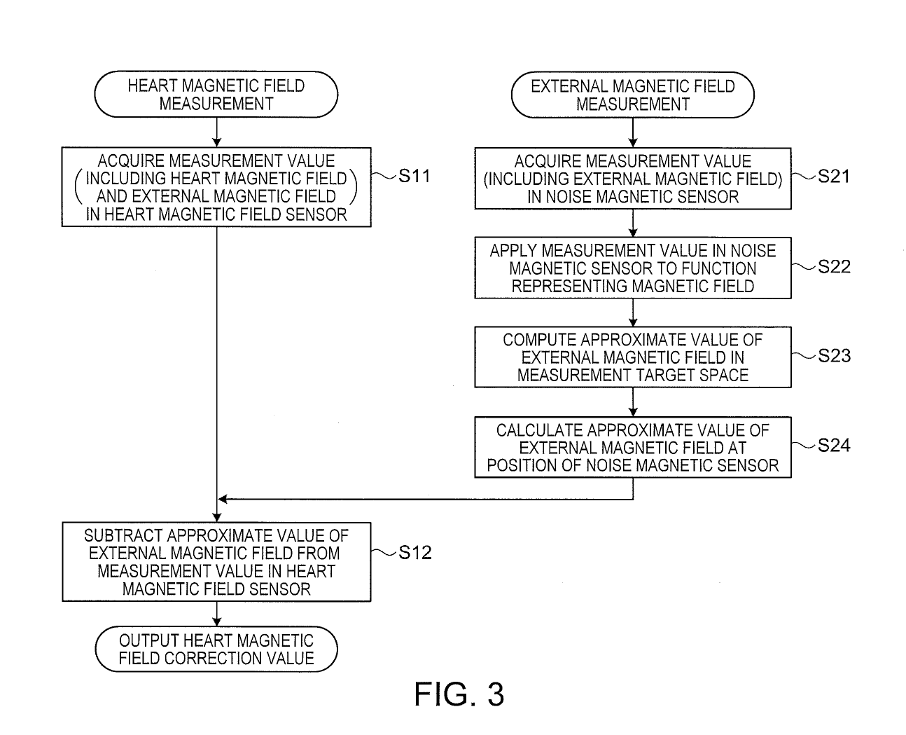

A description will be made of a schematic heart magnetic field measurement process performed by the controller of the magnetic measurement apparatus 2. FIG. 3 is a flowchart illustrating a schematic heart magnetic field measurement process according to the first embodiment. The left part in FIG. 3 illustrates a processing flow related to a measurement value in the heart magnetic field sensor 10, and the upper part in FIG. 3 illustrates a processing flow related to a measurement value in the noise magnetic sensor 30.

In step S11, the controller acquires a measurement value in the heart magnetic field sensor 10. The measurement value in the heart magnetic field sensor 10 includes a weak heart magnetic field (first magnetic field) and an external magnetic field (second magnetic field) such as magnetic noise. In step S21, the controller acquires a measurement value in the noise magnetic sensor 30. The measurement value in the noise magnetic sensor 30 includes an external magnetic field (second magnetic field) such as magnetic noise in the measurement target space including the position of the heart magnetic field sensor 10.

The acquisition of the measurement value in the heart magnetic field sensor 10 in step S11 and the acquisition of the measurement value in the noise magnetic sensor 30 in step S21 may be performed together, and may be performed separately. In a case where the noise magnetic sensor 30 detects not only an external magnetic field but also a heart magnetic field, that is, in a case where a measurement value in the noise magnetic sensor 30 includes a heart magnetic field and an external magnetic field, it is necessary to acquire a measurement value in the noise magnetic sensor 30 in step S21 in a state in which there is no subject 9 before acquiring a measurement value in the heart magnetic field sensor 10 in step S11.

In step S22, the controller applies the measurement value in the noise magnetic sensor 30 to a function representing a magnetic field. In step S22, it is preferable to use a function which can approximate a distribution of an external magnetic field in the measurement target space with high accuracy. Successively, in step S23, the controller computes an approximate value of the external magnetic field in the measurement target space. In step S24, the controller calculates an approximate value (also referred to as an approximate value vector A) of the external magnetic field at the position (also referred to as a measurement position) of the heart magnetic field sensor 10. A description will be made later of a method of computing an approximate value of the external magnetic field by applying the measurement value in the noise magnetic sensor 30 acquired in step S21 to the function in steps S22 to S24.

Next, in step S12, the controller subtracts the approximate value of the external magnetic field at the position of the heart magnetic field sensor 10, calculated in step S24, from the measurement value in the heart magnetic field sensor 10, acquired in step S11. Specifically, in a case where the heart magnetic field sensor 10 measures a magnetic field vector, an approximate value vector may be subtracted vectorially, or a specific component may be subtracted. The heart magnetic field sensor 10 according to the present embodiment measures a component (a Z component in the example of the present embodiment) of linearly polarized light in a traveling direction with high accuracy. In this case, the component of the approximate value may be subtracted.

Generally, in a case where a measurement direction in the heart magnetic field sensor 10 is set to correspond to a fourth vector d, since the heart magnetic field sensor 10 measures an inner product value B₀·d between a magnetic field B₀ at the location (measurement position) where the heart magnetic field sensor 10 is located and the fourth vector d, an inner product value A·d between an approximate value vector A at the measurement position and the fourth vector d may be subtracted from the measurement value (inner product value B₀·d) in the heart magnetic field sensor 10. As a result, the external magnetic field (second magnetic field) which is a noise component included in the measurement value obtained by the heart magnetic field sensor 10, and thus it is possible to obtain a measurement value of the heart magnetic field (first magnetic field) which is a signal component.

The magnetic measurement system 1 according to the present embodiment repeatedly performs the processing flow illustrated in FIG. 3 and can thus measure a temporally changing magnetic field. Therefore, in a configuration in which a calculation process can be performed at a high speed (for example, time resolution of 100 Hz), a magnetic field waveform can be acquired almost in a real time. In a case where it is hard to perform the calculation process at a high speed, acquired measurement values may be stored in a time series, and, after the measurement is completed, a calculation process on the stored measurement values may be performed.

### Method of Computing Approximate Value of External Magnetic Field

Next, a description will be made of a method of computing an approximate value of an external magnetic field. The controller of the magnetic measurement apparatus 2 applies the measurement value in the noise magnetic sensor 30 acquired in the above-described step S21 to a multi-variable polynomial so as to compute an approximate value of an external magnetic field in the measurement target space. In the present embodiment, a heart magnetic field which is the first magnetic field is weaker than an external magnetic field which is the second magnetic field.

Any position in the measurement target space is represented by a position vector r (hereinafter, also referred to as a position r) as shown in Equation (18).

$$\vec{r} = ( \begin{matrix}x \\ y \\ z\end{matrix} ) \qquad ( 18 )$$

A magnetic field at the position r is represented by a magnetic field vector B (hereinafter, also referred to as a magnetic field B) as shown in Equation (19).

$$\vec{B} ( \vec{r} ) = ( \begin{matrix}{B}_{x} ( \vec{r} ) \\ {B}_{y} ( \vec{r} ) \\ {B}_{z} ( \vec{r} )\end{matrix} ) ≡ ( \begin{matrix}{B}_{x} \\ {B}_{y} \\ {B}_{z}\end{matrix} ) = ( \begin{matrix}{B}_{1} \\ {B}_{2} \\ {B}_{3}\end{matrix} ) \qquad ( 19 )$$

According to the intensive examination performed by the present inventor, the magnetic field B has proved to be approximated with high accuracy in a multi-variable polynomial (which includes three variables and is a polynomial of a quadratic expression regarding the variables) shown in Equation (20). In Equation (20), aᵢⱼ (where i is an integer of 1 to 3, and j is an integer of 1 to 7) is a coefficient, x, y, and z are space coordinates of the approximate value B of the magnetic field, and Bᵢ is an i-th component of the approximate value B of the magnetic field. Equation (20) is also a non-linear polynomial (which includes three variables, and linear terms and quadratic terms regarding the variables).

Bᵢ=aᵢ₁+aᵢ₂x+aᵢ₃y+aᵢ₄z+aᵢ₅xy+aᵢ₆yz+aᵢ₇zx (20)

In Equation (20), i=1 indicates an X component Bₓ of the magnetic field B, i=2 indicates a Y component B_{y} of the magnetic field B, and i=3 indicates a Z component B_{z}, of the magnetic field B. Specifically, the respective components of the magnetic field B are expressed by Equation (21).

B₁=Bₓ=a₁₁+a₁₂x+a₁₃y+a₁₄z+a₁₅xy+a₁₆yz+a₁₇zx

B₂=B_{y}=a₂₁+a₂₂x+a₂₃y+a₂₄z+a₂₅xy+a₂₆yz+a₂₇zx

B₃=B_{z}=a₃₁+a₃₂x+a₃₃y+a₃₄z+a₃₅xy+a₃₆yz+a₃₇zx (21)

The first term (aᵢ₁) of the right side of the multi-variable polynomial shown in Equation (20) indicates a parallel magnetic field (deviation or offset magnetic field from the origin) in the entire measurement target space. The second term (aᵢ₂x) to the fourth term (aᵢ₄y) of the right side of the multi-variable polynomial shown in Equation (20) indicate a linear magnetic field (gradient of the magnetic field or a gradient magnetic field). The fifth term (aᵢ₅xy) to the seventh term (aᵢ₇zx) of the right side of the multi-variable polynomial shown in Equation (20) indicate an alternating magnetic field (torsion of the magnetic field or a rotating magnetic field).

A component which is proportional to an outer product term between a current element vector and a position vector is present in the magnetic field B according to the Biot-Savart law. Therefore, the present inventor has introduced an xy term, a yz term, and a zx term representing torsion into an approximate expression of the magnetic field B. For this reason, the magnetic field B at any position in the measurement target space is approximated with high accuracy by using the multi-variable polynomial shown in Equation (20).

In the multi-variable polynomial shown in Equation (20), seven unknowns aᵢⱼ (where j is an integer of 1 to 7) are present for each of three XYZ components, and thus a total of 21(=3×7) unknowns are present. The 21 unknowns are values specific to the measurement target space, and, if such unknowns corresponding to the measurement target space are specified, the magnetic field B in the measurement target space can be approximated with high accuracy. Next, a method of specifying the unknown aᵢⱼ will be described.

First, a noise magnetic sensor term vector R (hereinafter, also referred to as a magnetic sensor term vector R) is defined in Equation (22).

$$\vec{R} ( \vec{r} ) = ( \begin{matrix}1 \\ x \\ y \\ z \\ xy \\ yz \\ zx\end{matrix} ) ≡ ( \begin{matrix}{R}_{1} \\ {R}_{2} \\ {R}_{3} \\ {R}_{4} \\ {R}_{5} \\ {R}_{6} \\ {R}_{7}\end{matrix} ) \qquad ( 22 )$$

If the magnetic sensor term vector R of Equation (22) is used, the magnetic field vector B of Equation (21) is expressed as in Equation (23).

$$\vec{B} ( \vec{r} ) = ( \begin{matrix}{B}_{x} \\ {B}_{y} \\ {B}_{z}\end{matrix} ) = ( \begin{matrix}{a}_{11} & {a}_{12} & {a}_{13} & {a}_{14} & {a}_{15} & {a}_{16} & {a}_{17} \\ {a}_{21} & {a}_{22} & {a}_{23} & {a}_{24} & {a}_{25} & {a}_{26} & {a}_{27} \\ {a}_{31} & {a}_{32} & {a}_{33} & {a}_{34} & {a}_{35} & {a}_{36} & {a}_{37}\end{matrix} ) ( \begin{matrix}1 \\ x \\ y \\ z \\ xy \\ yz \\ zx\end{matrix} ) ≡ a \vec{R} \qquad ( 23 )$$

The last equal sign in Equation (23) indicates the definition of an unknown matrix a (hereinafter, also referred to as a first matrix a). Similarly, the matrix elements shown in Equation (20) are expressed by Equation (24).

$${B}_{i} = {\sum }_{j = 1}^{7} {a}_{ij} {R}_{j} \qquad ( 24 )$$

The number of noise magnetic sensors 30 is indicated by α. As described above, since 21 unknowns aᵢⱼ are present, in the present embodiment, α is an integer of 7 or greater, and α is 8 as an example. Since each noise magnetic sensor 30 measures the three XYZ components, if α is 7 or greater, at least 21 unknowns aᵢⱼ can be specified.

A position of a k-th noise magnetic sensor 30 is represented by a noise magnetic sensor position vector rₖ (hereinafter, also referred to as a magnetic sensor position rₖ) as shown in Equation (25). Here, k is an integer of 1 to α, and, in the example of the present embodiment, the number of noise magnetic sensors 30 is α=8, and thus k is an integer of 1 to 8.

$${\vec{r}}_{k} = ( \begin{matrix}{x}_{k} \\ {y}_{k} \\ {z}_{k}\end{matrix} ) ≡ ( \begin{matrix}{r}_{1 k} \\ {r}_{2 k} \\ {r}_{3 k}\end{matrix} ) \qquad ( 25 )$$

The magnetic field B at the position of the k-th noise magnetic sensor 30 (magnetic sensor position rₖ) is represented by a k-th detection magnetic field vector Bₖ (hereinafter, also referred to as a detection magnetic field Bₖ) as shown in Equation (26).

$$\vec{B} ( {\vec{r}}_{k} ) = ( \begin{matrix}{B}_{x} ( {\vec{r}}_{k} ) \\ {B}_{y} ( {\vec{r}}_{k} ) \\ {B}_{z} ( {\vec{r}}_{k} )\end{matrix} ) ≡ ( \begin{matrix}{B}_{xk} \\ {B}_{yk} \\ {B}_{zk}\end{matrix} ) = ( \begin{matrix}{B}_{1 k} \\ {B}_{2 k} \\ {B}_{3 k}\end{matrix} ) \qquad ( 26 )$$

If Equation (23) is applied to Equation (26), Equation (27) is obtained.

$$\vec{B} ( {\vec{r}}_{k} ) = ( \begin{matrix}{B}_{1 k} \\ {B}_{2 k} \\ {B}_{3 k}\end{matrix} ) = ( \begin{matrix}{a}_{11} & {a}_{12} & {a}_{13} & {a}_{14} & {a}_{15} & {a}_{16} & {a}_{17} \\ {a}_{21} & {a}_{22} & {a}_{23} & {a}_{24} & {a}_{25} & {a}_{26} & {a}_{27} \\ {a}_{31} & {a}_{32} & {a}_{33} & {a}_{34} & {a}_{35} & {a}_{36} & {a}_{37}\end{matrix} ) ( \begin{matrix}1 \\ {x}_{k} \\ {y}_{k} \\ {z}_{k} \\ {x}_{k} {y}_{k} \\ {y}_{k} {z}_{k} \\ {z}_{k} {x}_{k}\end{matrix} ) ≡ a {\vec{R}}_{k} = a ( \begin{matrix}{R}_{1 k} \\ {R}_{2 k} \\ {R}_{3 k} \\ {R}_{4 k} \\ {R}_{5 k} \\ {R}_{6 k} \\ {R}_{7 k}\end{matrix} ) \qquad ( 27 )$$

In Equation (27), the second to last equal sign indicates the definition of a magnetic sensor term vector Rₖ at the magnetic sensor position rₖ. In this case, a matrix element Bᵢₖ indicating an i-th row component of the detection magnetic field Bₖ which is shown in Equation (24) and is detected by the k-th noise magnetic sensor 30 is expressed by Equation (28).

$${B}_{ik} = {\sum }_{j = 1}^{7} {a}_{ij} {R}_{jk} \qquad ( 28 )$$

Next, by using Equation (27) or (28), a detection magnetic field matrix M (also referred to as a second matrix M) formed of all of a detection magnetic field vectors Bₖ, and a magnetic sensor term matrix P (also referred to as a third matrix P) formed of all of a magnetic sensor term vectors Rₖ are respectively expressed by Equation (29) and Equation (30).

$$\begin{matrix}M = ( {\vec{B}}_{1} ⋯ {\vec{B}}_{α} ) = ( \begin{matrix}{B}_{11} & {B}_{12} & … & {B}_{1 α} \\ {B}_{21} & {B}_{22} & … & {B}_{2 α} \\ {B}_{31} & {B}_{32} & … & {B}_{3 α}\end{matrix} ) & ( 29 ) \\ P = ( {\vec{R}}_{1} ⋯ {\vec{R}}_{α} ) = ( \begin{matrix}{R}_{11} & {R}_{12} & … & {R}_{1 α} \\ {R}_{21} & {R}_{22} & … & {R}_{2 α} \\ {R}_{31} & {R}_{32} & … & {R}_{3 α} \\ {R}_{41} & {R}_{42} & … & {R}_{4 α} \\ {R}_{51} & {R}_{52} & … & {R}_{5 α} \\ {R}_{61} & {R}_{62} & … & {R}_{6 α} \\ {R}_{71} & {R}_{72} & … & {R}_{7 α}\end{matrix} ) = ( \begin{matrix}1 & 1 & … & 1 \\ {x}_{1} & {x}_{2} & … & {x}_{α} \\ {y}_{1} & {y}_{2} & … & {y}_{α} \\ {z}_{1} & {z}_{2} & … & {z}_{α} \\ {x}_{1} {y}_{1} & {x}_{2} {y}_{2} & … & {x}_{α} {y}_{α} \\ {y}_{1} {z}_{1} & {y}_{2} {z}_{2} & … & {y}_{α} {z}_{α} \\ {z}_{1} {x}_{1} & {z}_{2} {x}_{2} & … & {z}_{α} {x}_{α}\end{matrix} ) & ( 30 )\end{matrix}$$

As illustrated in Equation (29), the detection magnetic field matrix M is a matrix of three rows and α columns, and a matrix element Mᵢₖ of i rows and k columns is expressed by Mᵢₖ=Bᵢₖ. In other words, the detection magnetic field matrix M is a matrix obtained by arranging the detection magnetic field vectors Bₖ of three rows and one column by α columns from k=1 to α. Therefore, for example, Bᵢₖ is an i-th row component of a magnetic field detected by the k-th noise magnetic sensor 30. As described above, i=1 indicates an X component, i=2 indicates a Y component, and i=3 indicates a Z component.

As shown in Equation (30), the magnetic sensor term matrix P is a matrix of seven rows and α columns, and a matrix element P_{gk} of g rows and k columns is expressed by P_{gk}=R_{gk}. In other words, the magnetic sensor term matrix P is obtained by arranging the magnetic sensor term vector Rₖ of seven rows and one column by α columns from k=1 to α.

Therefore, R_{gk} is a g-th row component of the magnetic sensor term vector Rₖ in the k-th noise magnetic sensor 30 (magnetic sensor position rₖ). For example, if g=2, R_{gk} is xₖ, and if g=7, R_{gk} is zₖxₖ. A relationship between the detection magnetic field matrix M (second matrix M) and the magnetic sensor term matrix P (third matrix P) is expressed by Equation (31) by using the unknown matrix a (first matrix a).

$$M = aP , \\ ( \begin{matrix}{B}_{11} & {B}_{12} & … & {B}_{1 α} \\ {B}_{21} & {B}_{22} & … & {B}_{2 α} \\ {B}_{31} & {B}_{32} & … & {B}_{3 α}\end{matrix} ) = ( \begin{matrix}{a}_{11} & {a}_{12} & {a}_{13} & {a}_{14} & {a}_{15} & {a}_{16} & {a}_{17} \\ {a}_{21} & {a}_{22} & {a}_{23} & {a}_{24} & {a}_{25} & {a}_{26} & {a}_{27} \\ {a}_{31} & {a}_{32} & {a}_{33} & {a}_{34} & {a}_{35} & {a}_{36} & {a}_{37}\end{matrix} ) ( \begin{matrix}{R}_{11} & {R}_{12} & … & {R}_{1 α} \\ {R}_{21} & {R}_{22} & … & {R}_{2 α} \\ {R}_{31} & {R}_{32} & … & {R}_{3 α} \\ {R}_{41} & {R}_{42} & … & {R}_{4 α} \\ {R}_{51} & {R}_{52} & … & {R}_{5 α} \\ {R}_{61} & {R}_{62} & … & {R}_{6 α} \\ {R}_{71} & {R}_{72} & … & {R}_{7 α}\end{matrix} ) \qquad ( 31 )$$

If Equation (31) is expressed by using matrix elements, this leads to Equation (32).

$${B}_{2 k} = {\sum }_{j = 1}^{7} {a}_{ij} {R}_{jk} \qquad ( 32 )$$

As described above, i is an integer of 1 to 3, and k indicating the noise magnetic sensors 30 is an integer of 1 to α. Therefore, for example, B₂ₖ which is a Y component (i=2) of the magnetic field B detected by the k-th noise magnetic sensor 30 is expressed by Equation (33).

$$\begin{matrix}{B}_{2 k} = {\sum }_{j = 1}^{7} {a}_{2 j} {R}_{jk} = {a}_{21} {R}_{1 k} + {a}_{22} {R}_{2 k} + {a}_{23} {R}_{3 k} + \\ {a}_{24} {R}_{4 k} + {a}_{25} {R}_{5 k} + {a}_{26} {R}_{6 k} + {a}_{27} {R}_{7 k} \\ = {a}_{21} + {a}_{22} {x}_{k} + {a}_{23} {y}_{k} + {a}_{24} {z}_{k} + {a}_{25} {x}_{k} {y}_{k} + \\ {a}_{26} {y}_{k} {z}_{k} + {a}_{27} {z}_{k} {y}_{k}\end{matrix} \qquad ( 33 )$$

The unknown matrix a (first matrix a) is to be obtained from Equation (31). If the number α of noise magnetic sensors 30 is 7, the magnetic sensor term matrix P (third matrix P) is a square matrix of seven rows and seven columns, and thus an inverse matrix thereof is present. In this case, as shown in Equation (34), the unknown matrix a (first matrix a) is obtained by multiplying the detection magnetic field matrix M (second matrix M) by an inverse matrix P⁻¹ of the magnetic sensor term matrix P (third matrix P) from the right.

a=MP⁻¹ (34)

On the other hand, if the number α of noise magnetic sensors 30 is 8 or larger, the magnetic sensor term matrix P (third matrix P) is not a square matrix, and thus an inverse matrix thereof is not present. In this case, as shown in Equation (35), the unknown matrix a (first matrix a) is obtained by multiplying the detection magnetic field matrix M (second matrix M) by a pseudo-inverse matrix (also referred to as a generalized inverse matrix) P⁺ of the magnetic sensor term matrix P (third matrix P) from the right.

a=MP⁺ (35)

In Equation (35), the pseudo-inverse matrix P⁺ of the magnetic sensor term matrix P (third matrix P) is obtained by using Equation (36).

P⁺=(P^{T}P)⁻¹P^{T} (36)

As shown in Equation (36), the pseudo-inverse matrix P⁺ is obtained by multiplying an inverse matrix of a product between a transposed matrix P^{T} of the magnetic sensor term matrix P and the magnetic sensor term matrix P by the transposed matrix P^{T} of the magnetic sensor term matrix P. The transposed matrix P^{T} of the magnetic sensor term matrix P is obtained by replacing matrix elements of the magnetic sensor term matrix P with respect to rows and columns, and is a matrix of a rows and seven columns as expressed by Equation (37).

$${P}^{T} = ( \begin{matrix}{R}_{11} & … & {P}_{71} \\ {R}_{12} & … & {P}_{72} \\ ⋮ & & ⋮ \\ {P}_{1 α} & … & {R}_{7 α}\end{matrix} ) \qquad ( 37 )$$

As mentioned above, if the pseudo-inverse matrix P⁺ is used, a principle of the least square method acts, and thus an optimal solution which minimizes errors in the unknown matrix a (first matrix a) is defined.

As mentioned above, the 21 unknowns in the multi-variable polynomial shown in Equation (20) are specified, and thus the magnetic field B at any location in the measurement target space can be approximated with high accuracy. As a result, it is possible to calculate an approximate value (approximate value vector A) of an external magnetic field at the position of the heart magnetic field sensor 10 with high accuracy.

Specifically, the unknown matrix a obtained by using Equation (34) or (35) is applied to Equation (23). At this time, coordinates of the heart magnetic field sensor 10 are applied to the magnetic sensor term vector R. As a result, the magnetic field vector B in Equation (23) represents the approximate value vector A at the position of the heart magnetic field sensor 10. The calculated approximate value (approximate value vector A) of the external magnetic field is removed from the measurement value in the heart magnetic field sensor 10, and thus a measurement target heart magnetic field can be measured with less noise. In other words, a weak signal such as a heart magnetic field can be extracted at a high signal to noise (S/N) ratio.

As mentioned above, according to the magnetic measurement system 1 of the present embodiment, even in a case where an external magnetic field (for example, magnetic noise) stronger than a measurement target magnetic field (for example, a heart magnetic field) is present, an external magnetic field at a measurement target position can be approximated with high accuracy through calculation and can be removed from a measurement value. Therefore, it is possible to measure a weak magnetic field such as a heart magnetic field with high accuracy.

As illustrated in FIG. 2B, if the heart magnetic field sensor 10 is constituted of a plurality of sensor elements 11 which are disposed in a matrix, there is a case where an optical axis of the laser light 18a which is incident to the gas cell 12 may vary in each sensor element 11. According to the magnetic measurement system 1 of the present embodiment, even if there is a variation in the optical axis of the laser light 18a which is incident to the gas cell 12 in each sensor element 11, an external magnetic field can be separately approximated and be removed for each sensor element 11, and thus measurement accuracy of a weak magnetic field can be maintained to be high.

According to the magnetic measurement system 1 of the present embodiment, the heart magnetic field measurement process is performed by using matrix calculation, and thus the controller of the magnetic measurement apparatus 2 can be constituted of a simple element such as a gate array. Consequently, it is possible to implement the magnetic measurement system 1 at lower cost.

Next, a description will be made of a method of computing the magnetic sensor term matrix P (third matrix P) in computation of an approximate value of an external magnetic field in the first embodiment by using Examples of arrangement of the noise magnetic sensors 30.

### Example 1-1

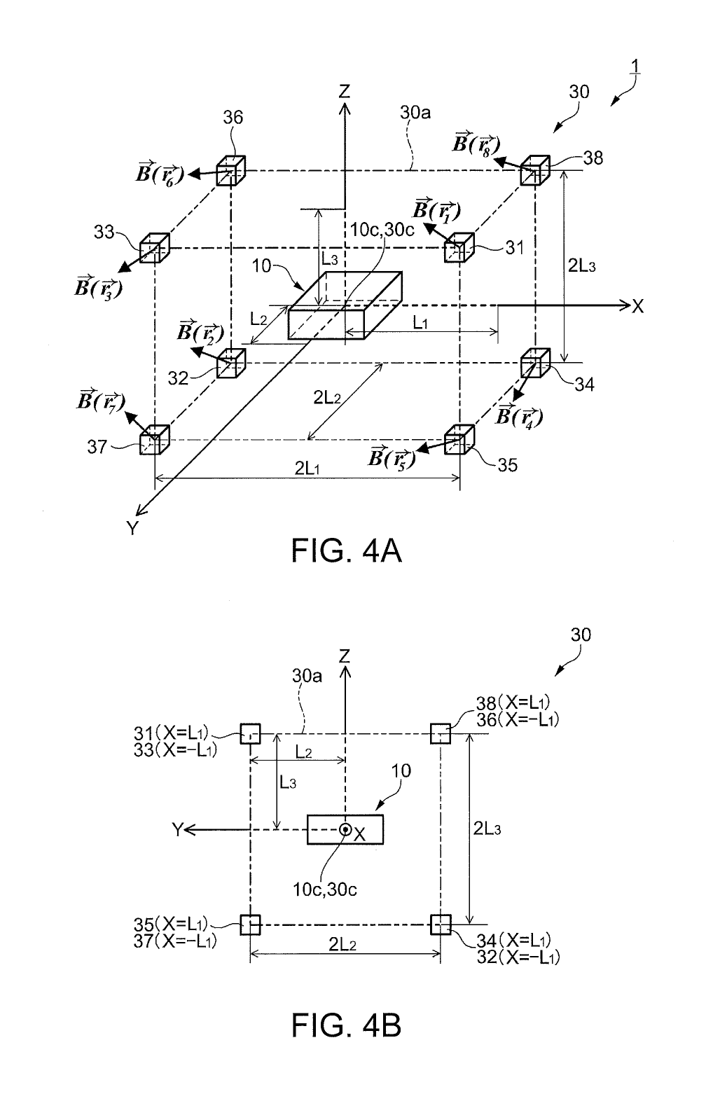

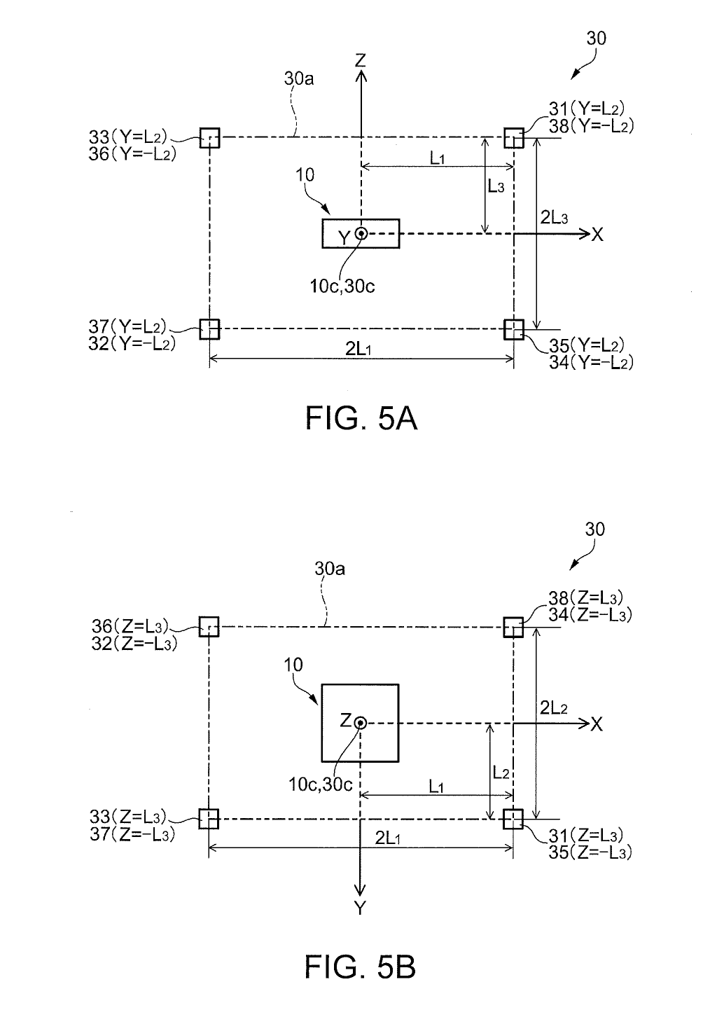

FIGS. 4A to 5B are diagrams illustrating arrangement of the noise magnetic sensors related to Example 1-1. Specifically, FIG. 4A is a perspective view, and FIG. 4B is a plan view which is viewed from the +X direction side in FIG. 4A. FIG. 5A is a plan view which is viewed from the +Y direction side in FIG. 4A, and FIG. 5B is a plan view which is viewed from the +Z direction side in FIG. 4A.

In FIGS. 4A to 5B, in order to individually identify eight noise magnetic sensors 30, first to eighth noise magnetic sensors are referred to as noise magnetic sensors 31, 32, 33, 34, 35, 36, 37 and 38. The noise magnetic sensors 31, 32, 33, 34, 35, 36, 37 and 38 are collectively referred to as noise magnetic sensors 30 in some cases.

In the magnetic measurement system 1, four sensors such as the noise magnetic sensors 31, 33, 36 and 38 are attached to, for example, the main body 6a (refer to FIG. 1), and are disposed at four corners of a plane which is parallel to the X-Y plane on an upper side of the measurement target space. Four sensors such as the noise magnetic sensors 32, 34, 35 and 37 are attached to, for example, the Y-direction table 4c (refer to FIG. 1), and are disposed at four corners of a plane which is parallel to the X-Y plane on a lower side of the measurement target space and overlaps the plane on which the noise magnetic sensors 31, 33, 36 and 38 are disposed in a plan view.

Therefore, as illustrated in FIG. 4A, the eight noise magnetic sensors 30 (31, 32, 33, 34, 35, 36, 37, and 38) are disposed at respective vertices of a rectangular parallelepiped 30a. The rectangular parallelepiped 30a is a hexahedron constituted of two planes parallel to the X-Y plane, two planes parallel to the Y-Z plane, and two planes parallel to the X-Z plane.

The eight noise magnetic sensors 30 are disposed so that the center 30c of the rectangular parallelepiped 30a substantially matches the center 10c of the heart magnetic field sensor 10. In the present example, it is assumed that the center 30c of the rectangular parallelepiped 30a matches the center 10c of the heart magnetic field sensor 10, and the center 30c of the rectangular parallelepiped 30a and the center 10c of the heart magnetic field sensor 10 are disposed at the origin of the XYZ orthogonal coordinate system in FIGS. 4A and 4B.

As mentioned above, 2n (where n is an integer of 3 or greater) noise magnetic sensors 30 are formed as n pairs of noise magnetic sensors 30, and the heart magnetic field sensor 10 is disposed at the centroid position of the noise magnetic sensors 30 forming each pair for each pair. In the above-described way, measurement values from each pair contribute to the approximate value vector A at the position of the heart magnetic field sensor 10 with the same importance. If lengths of the respective pairs are aligned, the importances of measurement values from the 2n (where n is an integer of 3 or greater) noise magnetic sensors 30 are the same as each other, and thus it is possible to measure the approximate value vector A at the position of the heart magnetic field sensor 10 with higher accuracy.

A length of a side of the rectangular parallelepiped 30a along the X axis is indicated by 2L₁, a length of a side of the rectangular parallelepiped 30a along the Y axis is indicated by 2L₂, and a length of a side of the rectangular parallelepiped 30a along the Z axis is indicated by 2L₃. As illustrated in FIGS. 4A and 4B, the first noise magnetic sensor 31, the fourth noise magnetic sensor 34, the fifth noise magnetic sensor 35, and the eighth noise magnetic sensor 38 are disposed in a plane of X=L₁ parallel to the Y-Z plane. The second noise magnetic sensor 32, the third noise magnetic sensor 33, the sixth noise magnetic sensor 36, and the seventh noise magnetic sensor 37 are disposed in a plane of X=−L₁ parallel to the Y-Z plane.

As illustrated in FIGS. 4A and 5A, the first noise magnetic sensor 31, the third noise magnetic sensor 33, the fifth noise magnetic sensor 35, and the seventh noise magnetic sensor 37 are disposed in a plane of Y=L₂ parallel to the X-Z plane. The second noise magnetic sensor 32, the fourth noise magnetic sensor 34, the sixth noise magnetic sensor 36, and the eighth noise magnetic sensor 38 are disposed in a plane of Y=−L₂ parallel to the X-Z plane.

As illustrated in FIGS. 4A and 5B, the first noise magnetic sensor 31, the third noise magnetic sensor 33, the sixth noise magnetic sensor 36, and the eighth noise magnetic sensor 38 are disposed in a plane of Z=L₃ parallel to the X-Y plane. The second noise magnetic sensor 32, the fourth noise magnetic sensor 34, the fifth noise magnetic sensor 35, and the seventh noise magnetic sensor 37 are disposed in a plane of Z=−L₃ parallel to the X-Y plane.

When the eight noise magnetic sensors 30 are disposed, the most preferable rectangular parallelepiped 30a is a rectangular parallelepiped 30a with L₁/2^{1/2}=L₂=L₃=L. In other words, the rectangular parallelepiped 30a is a quadrangular prism which has a square (Y-Z section) of which a length of one side is 2L as a bottom and has a height (a length of a side along the X axis) of 2×2^{1/2}×L. In the rectangular parallelepiped 30a, a normal direction of the bottom is parallel to the X axis, normal directions of two side surfaces are parallel to the Y axis, and normal directions of the two remaining side surfaces are parallel to the Z axis. A position vector rₖ (magnetic sensor position rₖ) of the noise magnetic sensors 30 disposed at respective vertices of the rectangular parallelepiped 30a is expressed by Equation (38).

$${\vec{r}}_{1} = L ( \begin{matrix}\sqrt{2} \\ 1 \\ 1\end{matrix} ) {\vec{r}}_{2} = L ( \begin{matrix}- \sqrt{2} \\ - 1 \\ - 1\end{matrix} ) {\vec{r}}_{3} = L ( \begin{matrix}- \sqrt{2} \\ 1 \\ 1\end{matrix} ) {\vec{r}}_{4} = L ( \begin{matrix}\sqrt{2} \\ - 1 \\ - 1\end{matrix} ) \\ {\vec{r}}_{5} = L ( \begin{matrix}\sqrt{2} \\ 1 \\ - 1\end{matrix} ) {\vec{r}}_{6} = L ( \begin{matrix}- \sqrt{2} \\ - 1 \\ 1\end{matrix} ) {\vec{r}}_{7} = ( \begin{matrix}- \sqrt{2} \\ 1 \\ - 1\end{matrix} ) {\vec{r}}_{8} = L ( \begin{matrix}\sqrt{2} \\ - 1 \\ 1\end{matrix} ) \\ \qquad ( 38 )$$

If the eight noise magnetic sensors 30 are disposed in the above-described manner, the magnetic sensor term matrix P (third matrix P) is expressed by Equation (39), and thus computation is relatively simplified. In this case, since a line segment connecting the first noise magnetic sensor 31 to the second noise magnetic sensor 32 is orthogonal to a line segment connecting the third noise magnetic sensor 33 to the fourth noise magnetic sensor 34, an approximate value of the second magnetic field around the origin (around the position of the heart magnetic field sensor 10) can be computed with high accuracy. Similarly, a line segment connecting the fifth noise magnetic sensor 35 to the sixth noise magnetic sensor 36 is orthogonal to a line segment connecting the seventh noise magnetic sensor 37 to the eighth noise magnetic sensor 38.

$$P = ( \begin{matrix}1 & 1 & 1 & 1 & 1 & 1 & 1 & 1 \\ \sqrt{2} L & - \sqrt{2} L & - \sqrt{2} L & \sqrt{2} L & \sqrt{2} L & - \sqrt{2} L & - \sqrt{2} L & \sqrt{2} L \\ L & - L & L & - L & L & - L & L & - L \\ L & - L & L & - L & - L & L & - L & L \\ \sqrt{2} {L}^{2} & \sqrt{2} {L}^{2} & - \sqrt{2} {L}^{2} & - \sqrt{2} {L}^{2} & \sqrt{2} {L}^{2} & \sqrt{2} {L}^{2} & - \sqrt{2} {L}^{2} & - \sqrt{2} {L}^{2} \\ {L}^{2} & {L}^{2} & {L}^{2} & {L}^{2} & - {L}^{2} & - {L}^{2} & - {L}^{2} & - {L}^{2} \\ \sqrt{2} {L}^{2} & \sqrt{2} {L}^{2} & - \sqrt{2} {L}^{2} & - \sqrt{2} {L}^{2} & - \sqrt{2} {L}^{2} & - \sqrt{2} {L}^{2} & \sqrt{2} {L}^{2} & \sqrt{2} {L}^{2}\end{matrix} ) \qquad ( 39 )$$

If L is used as one unit of the XYZ coordinate system, the magnetic sensor term matrix P (third matrix P) is expressed with 1 and −1 as shown in Equation (40), and thus computation is further simplified.

$$P = ( \begin{matrix}1 & 1 & 1 & 1 & 1 & 1 & 1 & 1 \\ \sqrt{2} & - \sqrt{2} & - \sqrt{2} & \sqrt{2} & \sqrt{2} & - \sqrt{2} & - \sqrt{2} & \sqrt{2} \\ 1 & - 1 & 1 & - 1 & 1 & - 1 & 1 & - 1 \\ 1 & - 1 & 1 & - 1 & - 1 & 1 & - 1 & 1 \\ \sqrt{2} & \sqrt{2} & - \sqrt{2} & - \sqrt{2} & \sqrt{2} & \sqrt{2} & - \sqrt{2} & - \sqrt{2} \\ 1 & 1 & 1 & 1 & - 1 & - 1 & - 1 & - 1 \\ \sqrt{2} & \sqrt{2} & - \sqrt{2} & - \sqrt{2} & - \sqrt{2} & - \sqrt{2} & \sqrt{2} & \sqrt{2}\end{matrix} ) \qquad ( 40 )$$

### Example 1-2

In Example 1-2, a positional relationship between the eight noise magnetic sensors 30 and the heart magnetic field sensor 10 is the same as in Example 1-1 and thus is not illustrated, but the most preferable rectangular parallelepiped 30a is a rectangular parallelepiped 30a with L₁=L₂=L₃=L. In other words, the rectangular parallelepiped 30a is a cube of which a length of one side is 2L. A position vector rₖ (magnetic sensor position rₖ) of the noise magnetic sensor 30 disposed at respective vertices of the cube of which a length of one side is 2L is expressed by Equation (41).

$${\vec{r}}_{1} = L ( \begin{matrix}1 \\ 1 \\ 1\end{matrix} ) {\vec{r}}_{2} = L ( \begin{matrix}- 1 \\ - 1 \\ - 1\end{matrix} ) {\vec{r}}_{3} = L ( \begin{matrix}- 1 \\ 1 \\ 1\end{matrix} ) {\vec{r}}_{4} = L ( \begin{matrix}1 \\ - 1 \\ - 1\end{matrix} ) \\ {\vec{r}}_{5} = L ( \begin{matrix}1 \\ 1 \\ - 1\end{matrix} ) {\vec{r}}_{6} = L ( \begin{matrix}- 1 \\ - 1 \\ 1\end{matrix} ) {\vec{r}}_{7} = L ( \begin{matrix}- 1 \\ 1 \\ - 1\end{matrix} ) {\vec{r}}_{8} = L ( \begin{matrix}1 \\ - 1 \\ 1\end{matrix} ) \qquad ( 41 )$$

If the eight noise magnetic sensors 30 are disposed in the above-described manner, the magnetic sensor term matrix P (third matrix P) is expressed with 1, L, and −L as shown in Equation (42), and thus computation is simplified.

$$P = ( \begin{matrix}1 & 1 & 1 & 1 & 1 & 1 & 1 & 1 \\ L & - L & - L & L & L & - L & - L & L \\ L & - L & L & - L & L & - L & L & - L \\ L & - L & L & - L & - L & L & - L & L \\ {L}^{2} & {L}^{2} & - {L}^{2} & - {L}^{2} & {L}^{2} & {L}^{2} & - {L}^{2} & - {L}^{2} \\ {L}^{2} & {L}^{2} & {L}^{2} & {L}^{2} & - {L}^{2} & - {L}^{2} & - {L}^{2} & - {L}^{2} \\ {L}^{2} & {L}^{2} & - {L}^{2} & - {L}^{2} & - {L}^{2} & - {L}^{2} & {L}^{2} & {L}^{2}\end{matrix} ) \qquad ( 42 )$$

If L is used as one unit of the XYZ coordinate system, the magnetic sensor term matrix P (third matrix P) is expressed with 1 and −1 as shown in Equation (43), and thus computation is further simplified.

$$P = ( \begin{matrix}1 & 1 & 1 & 1 & 1 & 1 & 1 & 1 \\ 1 & - 1 & - 1 & 1 & 1 & - 1 & - 1 & 1 \\ 1 & - 1 & 1 & - 1 & 1 & - 1 & 1 & - 1 \\ 1 & - 1 & 1 & - 1 & - 1 & 1 & - 1 & 1 \\ 1 & 1 & - 1 & - 1 & 1 & 1 & - 1 & - 1 \\ 1 & 1 & 1 & 1 & - 1 & - 1 & - 1 & - 1 \\ 1 & 1 & - 1 & - 1 & - 1 & - 1 & 1 & 1\end{matrix} ) \qquad ( 43 )$$

### Example 1-3

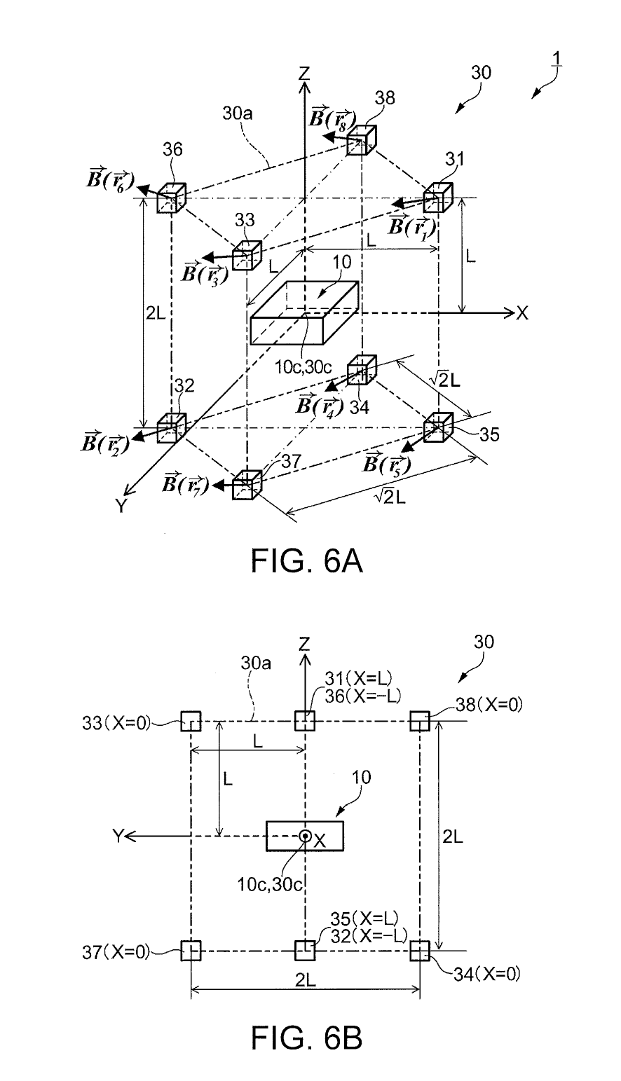

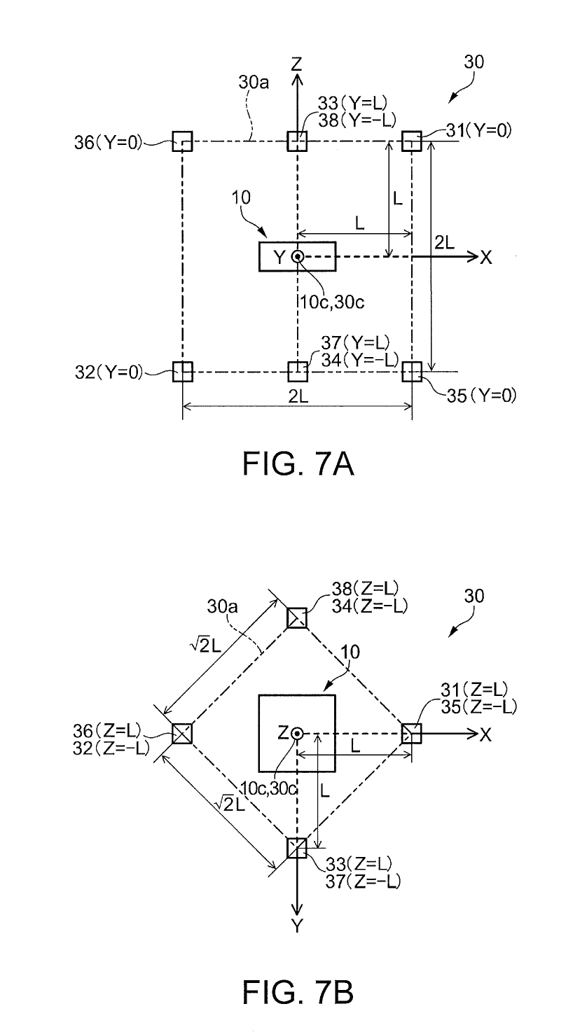

FIGS. 6A to 7B are diagrams illustrating arrangement of the noise magnetic sensors related to Example 1-3. Specifically, FIG. 6A is a perspective view, and FIG. 6B is a plan view which is viewed from the +X direction side in FIG. 6A. FIG. 7A is a plan view which is viewed from the +Y direction side in FIG. 6A, and FIG. 7B is a plan view which is viewed from the +Z direction side in FIG. 6A.

In Example 1-3, as illustrated in FIGS. 6A and 7B, the rectangular parallelepiped 30a is a quadrangular prism which has a square (X-Y section) of which a length of one side is 2^{1/2}×L as a bottom and has a height (a length of a side along the Z axis) of 2×L. A positional relationship between the eight noise magnetic sensors 30 (rectangular parallelepiped 30a) and the heart magnetic field sensor 10 is different from those in Example 1-1 and Example 1-2. More specifically, as illustrated in FIG. 7B, in a plan view from the +Z direction side, the rectangular parallelepiped 30a has a positional relationship of being rotated by 45° with the Z axis as the center relative to the heart magnetic field sensor 10. The eight noise magnetic sensors 30 are respectively disposed at the vertices of the rectangular parallelepiped 30a, and the center 30c of the rectangular parallelepiped 30a substantially matches the center 10c of the heart magnetic field sensor 10.

In Example 1-3, as illustrated in FIGS. 6A and 6B, among the eight noise magnetic sensors 30, four sensors such as the third noise magnetic sensor 33, the fourth noise magnetic sensor 34, the seventh noise magnetic sensor 37, and the eighth noise magnetic sensor 38 are disposed in a plane of X=0 parallel to the Y-Z plane. The four noise magnetic sensors 30 are respectively disposed at vertices of a square (also referred to as a first square) in the plane of X=0. In a plan view illustrated in FIG. 6B, the centroid of the first square is disposed to substantially match the origin, and the heart magnetic field sensor 10 is disposed to include the centroid of the first square and the origin.

As illustrated in FIGS. 6A and 7A, four sensors such as the first noise magnetic sensor 31, the second noise magnetic sensor 32, the fifth noise magnetic sensor 35, and the sixth noise magnetic sensor 36 are disposed in a plane of Y=0 parallel to the X-Z plane. The four noise magnetic sensors 30 are respectively disposed at vertices of a square (also referred to as a second square) in the plane of Y=0. In a plan view illustrated in FIG. 7A, the centroid of the second square is disposed to substantially match the origin, and the heart magnetic field sensor 10 is disposed to include the centroid of the second square and the origin.

A position vector rₖ (magnetic sensor position rₖ) of the noise magnetic sensors 30 disposed in the above-described manner is expressed by Equation (44).

$${\vec{r}}_{1} = L ( \begin{matrix}1 \\ 0 \\ 1\end{matrix} ) {\vec{r}}_{2} = L ( \begin{matrix}- 1 \\ 0 \\ - 1\end{matrix} ) {\vec{r}}_{3} = L ( \begin{matrix}0 \\ 1 \\ 1\end{matrix} ) {\vec{r}}_{4} = L ( \begin{matrix}0 \\ - 1 \\ - 1\end{matrix} ) \\ {\vec{r}}_{5} = L ( \begin{matrix}1 \\ 0 \\ - 1\end{matrix} ) {\vec{r}}_{6} = L ( \begin{matrix}- 1 \\ 0 \\ 1\end{matrix} ) {\vec{r}}_{7} = L ( \begin{matrix}0 \\ 1 \\ - 1\end{matrix} ) {\vec{r}}_{8} = L ( \begin{matrix}0 \\ - 1 \\ 1\end{matrix} ) \qquad ( 44 )$$

If the eight noise magnetic sensors 30 are disposed in the above-described manner, the magnetic sensor term matrix P (third matrix P) is expressed with 1, L, and −L as shown in Equation (45), and the number of zero matrix elements increases. Thus, computation is further simplified.

$$P = ( \begin{matrix}1 & 1 & 1 & 1 & 1 & 1 & 1 & 1 \\ L & - L & 0 & 0 & L & - L & 0 & 0 \\ 0 & 0 & L & - L & 0 & 0 & L & - L \\ L & - L & L & - L & - L & L & - L & L \\ 0 & 0 & 0 & 0 & 0 & 0 & 0 & 0 \\ 0 & 0 & {L}^{2} & {L}^{2} & 0 & 0 & - {L}^{2} & {L}^{2} \\ {L}^{2} & {L}^{2} & 0 & 0 & - {L}^{2} & - {L}^{2} & 0 & 0\end{matrix} ) \qquad ( 45 )$$

If L is used as one unit of the XYZ coordinate system, the magnetic sensor term matrix P (third matrix P) is expressed with 1 and −1 as shown in Equation (46), and thus computation is still further simplified.

$$P = ( \begin{matrix}1 & 1 & 1 & 1 & 1 & 1 & 1 & 1 \\ 1 & - 1 & 0 & 0 & 1 & - 1 & 0 & 0 \\ 0 & 0 & 1 & - 1 & 0 & 0 & 1 & - 1 \\ 1 & - 1 & 1 & - 1 & - 1 & 1 & - 1 & 1 \\ 0 & 0 & 0 & 0 & 0 & 0 & 0 & 0 \\ 0 & 0 & 1 & 1 & 0 & 0 & - 1 & - 1 \\ 1 & 1 & 0 & 0 & - 1 & - 1 & 0 & 0\end{matrix} ) \qquad ( 46 )$$

As mentioned above, according to the magnetic measurement system 1 of the first embodiment, even in a case where an external magnetic field stronger than a weak magnetic field such as a heart magnetic field is present, an external magnetic field at a measurement target position can be approximated with high accuracy through calculation and can be removed from a measurement value. Therefore, it is possible to measure a measurement target weak magnetic field with high accuracy.

### Second Embodiment

Next, a description will be made of a method of computing an approximate value of an external magnetic field in a magnetic measurement system according to a second embodiment. A magnetic measurement system according to the second embodiment is substantially the same as the first embodiment including a system configuration except that a method of expressing an unknown matrix in computation of an approximate value of an external magnetic field is different from that in the first embodiment.

### Method of Computing Approximate Value of External Magnetic Field

In the first embodiment, in computation of an approximate value of an external magnetic field, the unknown matrix a expressed by Equation (23) is defined on the basis of the unknown aᵢⱼ, and the unknown matrix a is solved by using Equation (34) or (35). In contrast, in the second embodiment, there is a difference in which an unknown vector b (also referred to as a first vector b) expressed by Equation (47) is defined on the basis of the unknown aᵢⱼ, and the unknown vector b is solved.

$$\vec{b} ≡ ( \begin{matrix}{a}_{11} \\ {a}_{12} \\ ⋮ \\ {a}_{16} \\ {a}_{17} \\ {a}_{21} \\ ⋮ \\ {a}_{27} \\ {a}_{31} \\ ⋮ \\ {a}_{37}\end{matrix} ) = ( \begin{matrix}{b}_{1} \\ {b}_{2} \\ ⋮ \\ {b}_{6} \\ {b}_{7} \\ {b}_{8} \\ ⋮ \\ {b}_{14} \\ {b}_{15} \\ ⋮ \\ {b}_{21}\end{matrix} ) \qquad ( 47 )$$

Hereinafter, a description will be made of a method of solving the unknown vector b in the magnetic measurement system according to the second embodiment. As illustrated in Equation (47), the unknown vector b (first vector b) is a column vector of twenty-one rows and one column in which 21 unknowns aᵢⱼ are arranged in one column. Next, a detection magnetic field vector N (also referred to as a second vector N) formed of all detection magnetic field vectors Bₖ in a noise magnetic sensors 30 (magnetic sensor position rₖ) is expressed by Equation (48).

$$\vec{N} ≡ ( \begin{matrix}{B}_{11} \\ {B}_{12} \\ {B}_{31} \\ {B}_{12} \\ {B}_{22} \\ {B}_{32} \\ ⋮ \\ {B}_{3 α - 1} \\ {B}_{1 α} \\ {B}_{2 α} \\ {B}_{3 α}\end{matrix} ) = ( \begin{matrix}{n}_{1} \\ {n}_{2} \\ {n}_{3} \\ {n}_{4} \\ {n}_{5} \\ {n}_{6} \\ ⋮ \\ {n}_{3 α - 3} \\ {n}_{3 α - 2} \\ {n}_{3 α - 1} \\ {n}_{3 α}\end{matrix} ) \qquad ( 48 )$$

As shown in Equation (48), the detection magnetic field vector N (second vector N) is a column vector of 3α rows and one column in which 3×α detection magnetic field matrix elements Bᵢₖ are arranged in one column Next, a magnetic sensor term matrix Q (also referred to as a fourth matrix Q) formed of all of a magnetic sensor term vectors Rₖ is expressed by Equation (49).

$$\begin{matrix}Q ≡ ( \begin{matrix}{\vec{R}}_{1}^{T} & \vec{0} & \vec{0} \\ \vec{0} & {\vec{R}}_{1}^{T} & \vec{0} \\ \vec{0} & \vec{0} & {\vec{R}}_{1}^{T} \\ {\vec{R}}_{2}^{T} & \vec{0} & \vec{0} \\ \vec{0} & {\vec{R}}_{2}^{T} & \vec{0} \\ \vec{0} & \vec{0} & {\vec{R}}_{2}^{T} \\ \vec{0} & \vec{0} & {\vec{R}}_{a - 1}^{T} \\ {\vec{R}}_{a}^{T} & \vec{0} & \vec{0} \\ \vec{0} & {\vec{R}}_{a}^{T} & \vec{0} \\ \vec{0} & \vec{0} & {\vec{R}}_{a}^{T}\end{matrix} ) \\ = ( \begin{matrix}{R}_{11} & {R}_{12} & ⋯ & {R}_{61} & {R}_{71} & 0 & 0 & ⋯ & 0 & 0 & 0 & 0 & ⋯ & 0 & 0 \\ 0 & 0 & ⋯ & 0 & 0 & {R}_{11} & {R}_{21} & ⋯ & {R}_{61} & {R}_{71} & 0 & 0 & ⋯ & 0 & 0 \\ 0 & 0 & ⋯ & 0 & 0 & 0 & 0 & ⋯ & 0 & 0 & {R}_{11} & {R}_{21} & ⋯ & {R}_{61} & {R}_{71} \\ {R}_{12} & {R}_{22} & ⋯ & {R}_{62} & {R}_{72} & 0 & 0 & ⋯ & 0 & 0 & 0 & 0 & ⋯ & 0 & 0 \\ 0 & 0 & ⋯ & 0 & 0 & {R}_{12} & {R}_{22} & ⋯ & {R}_{62} & {R}_{72} & 0 & 0 & ⋯ & 0 & 0 \\ 0 & 0 & ⋯ & 0 & 0 & 0 & 0 & ⋯ & 0 & 0 & {R}_{12} & {R}_{22} & ⋯ & {R}_{62} & {R}_{72} \\ ⋮ & ⋮ & & ⋮ & ⋮ & ⋮ & ⋮ & & ⋮ & ⋮ & ⋮ & ⋮ & & ⋮ & ⋮ \\ 0 & 0 & ⋯ & 0 & 0 & 0 & 0 & ⋯ & 0 & 0 & {R}_{1 a - 1} & {R}_{2 a - 1} & ⋯ & {R}_{6 a - 1} & {R}_{7 a - 1} \\ {R}_{1 a} & {R}_{2 a} & ⋯ & {R}_{6 a} & {R}_{7 a} & 0 & 0 & ⋯ & 0 & 0 & 0 & 0 & ⋯ & 0 & 0 \\ 0 & 0 & ⋯ & 0 & 0 & {R}_{1 a} & {R}_{2 a} & ⋯ & {R}_{6 a} & {R}_{7 a} & 0 & 0 & ⋯ & 0 & 0 \\ 0 & 0 & ⋯ & 0 & 0 & 0 & 0 & ⋯ & 0 & 0 & {R}_{1 a} & {R}_{2 a} & ⋯ & {R}_{6 a} & {R}_{7 a}\end{matrix} )\end{matrix} \qquad ( 49 )$$

In Equation (49), a column vector Rₖ^{T} is a transposed matrix of the magnetic sensor term vector Rₖ, and is a row vector of one row and seven columns. In Equation (49), a zero vector 0 is a row vector of one row and seven columns in which all matrix elements are zeros. Therefore, the fourth matrix Q is a matrix of 3α rows and twenty-one columns. If the fourth matrix Q defined in Equation (49) is used, the detection magnetic field vector N and the unknown vector b are expressed by Equation (50).

$$\vec{N} = Q \vec{b} , ( \begin{matrix}{B}_{11} \\ {B}_{21} \\ {B}_{31} \\ {B}_{12} \\ {B}_{22} \\ {B}_{32} \\ ⋮ \\ {B}_{3 a - 1} \\ {B}_{1 a} \\ {B}_{2 a} \\ {B}_{3 a}\end{matrix} ) = ( \begin{matrix}1 & {x}_{1} & ⋯ & {y}_{1} {z}_{1} & {z}_{1} {x}_{1} & 0 & 0 & ⋯ & 0 & 0 & 0 & 0 & ⋯ & 0 & 0 \\ 0 & 0 & ⋯ & 0 & 0 & 1 & {x}_{1} & ⋯ & {y}_{1} {z}_{1} & {z}_{1} {x}_{1} & 0 & 0 & ⋯ & 0 & 0 \\ 0 & 0 & ⋯ & 0 & 0 & 0 & 0 & ⋯ & 0 & 0 & 1 & {x}_{1} & ⋯ & {y}_{1} {z}_{1} & {z}_{1} {x}_{1} \\ 1 & {x}_{2} & ⋯ & {y}_{2} {z}_{2} & {z}_{2} {x}_{2} & 0 & 0 & ⋯ & 0 & 0 & 0 & 0 & ⋯ & 0 & 0 \\ 0 & 0 & ⋯ & 0 & 0 & 1 & {x}_{2} & ⋯ & {y}_{2} {x}_{2} & {z}_{2} {x}_{2} & 0 & 0 & ⋯ & 0 & 0 \\ 0 & 0 & ⋯ & 0 & 0 & 0 & 0 & ⋯ & 0 & 0 & 1 & {x}_{2} & ⋯ & {y}_{2} {z}_{2} & {z}_{2} {x}_{2} \\ ⋮ & ⋮ & & ⋮ & ⋮ & ⋮ & ⋮ & & ⋮ & ⋮ & ⋮ & ⋮ & & ⋮ & ⋮ \\ 0 & 0 & ⋯ & 0 & 0 & 0 & 0 & ⋯ & 0 & 0 & 1 & {z}_{a - 1} & ⋯ & {y}_{a - 1} {z}_{a - 1} & {z}_{a - 1} {x}_{a - 1} \\ 1 & {x}_{a} & ⋯ & {y}_{a} {z}_{a} & {z}_{a} {x}_{a} & 0 & 0 & ⋯ & 0 & 0 & 0 & 0 & ⋯ & 0 & 0 \\ 0 & 0 & ⋯ & 0 & 0 & 1 & {x}_{a} & ⋯ & {y}_{a} {z}_{a} & {z}_{a} {x}_{a} & 0 & 0 & ⋯ & 0 & 0 \\ 0 & 0 & ⋯ & 0 & 0 & 0 & 0 & ⋯ & 0 & 0 & 1 & {x}_{a} & ⋯ & {y}_{a} {z}_{a} & {z}_{a} {x}_{a}\end{matrix} ) ( \begin{matrix}{a}_{11} \\ {a}_{12} \\ ⋮ \\ {a}_{16} \\ {a}_{17} \\ {a}_{21} \\ {a}_{22} \\ ⋮ \\ {a}_{26} \\ {a}_{27} \\ {a}_{31} \\ {a}_{32} \\ ⋮ \\ {a}_{36} \\ {a}_{37}\end{matrix} ) \qquad ( 50 )$$

Here, in the same manner as in the first embodiment, if Equation (50) is expressed by using matrix elements, this leads to Equation (32). Therefore, it can be seen that Equation (50) represents the same equation system as in the first embodiment.

The unknown vector b is to be obtained from Equation (50). If the number α of noise magnetic sensors 30 is 7, the magnetic sensor term matrix Q (fourth matrix Q) is a square matrix of twenty-one rows and twenty-one columns, and thus an inverse matrix thereof is present. In this case, as shown in Equation (51), the unknown vector b (first vector b) is obtained by multiplying the detection magnetic field vector N (second vector N) by an inverse matrix Q⁻¹ of the fourth matrix Q from the left.

b̅=Q⁻¹N̅ (51)

On the other hand, if the number α of noise magnetic sensors 30 is 8 or larger, the magnetic sensor term matrix Q is not a square matrix, and thus an inverse matrix thereof is not present. In this case, as shown in Equation (52), the unknown vector b (first vector b) is obtained by multiplying the detection magnetic field vector N (second vector N) by a pseudo-inverse matrix (also referred to as a generalized inverse matrix) Q⁺ of the fourth matrix Q from the left.

b̅=Q⁺N̅ (52)

In Equation (52), the pseudo-inverse matrix Q⁺ of the magnetic sensor term matrix Q (fourth matrix Q) is obtained by using Equation (53).)

Q⁺=(Q^{T}Q)⁻¹Q^{T} (53)

As shown in Equation (53), the pseudo-inverse matrix Q⁺ is obtained by multiplying an inverse matrix of a product between a transposed matrix Q^{T} of the fourth matrix Q and the fourth matrix Q by the transposed matrix Q^{T} of the fourth matrix Q. The transposed matrix Q^{T} of the fourth matrix Q is obtained by replacing matrix elements of the fourth matrix Q with respect to rows and columns, and is a matrix of twenty-one rows and 3α columns as expressed by Equation (54).

$${Q}^{T} = ( \begin{matrix}{R}_{11} & 0 & 0 & {r}_{12} & ⋯ & ⋯ & 0 \\ {R}_{21} & 0 & 0 & {R}_{22} & ⋯ & ⋯ & 0 \\ ⋮ & & & & & & ⋮ \\ ⋮ & & & & & & ⋮ \\ 0 & 0 & {R}_{61} & 0 & ⋯ & ⋯ & {r}_{6 a} \\ 0 & 0 & {R}_{71} & 0 & ⋯ & ⋯ & {R}_{7 a}\end{matrix} ) \qquad ( 54 )$$

If the pseudo-inverse matrix Q⁺ is used to obtain the unknown vector b, a principle of the least square method acts, and thus an optimal solution which minimizes errors is defined. As mentioned above, in the second embodiment, since the least square method is applied to the whole unknown vector b, a more appropriate solution than the optimal solution obtained in the first embodiment can be obtained, and thus it is possible to more accurately approximate a magnetic field in the measurement target space.

### Third Embodiment

Next, a description will be made of a method of computing an approximate value of an external magnetic field in a magnetic measurement system according to a third embodiment. A magnetic measurement system according to the third embodiment has the same configuration as that in the first embodiment, and is substantially the same as the second embodiment except that a method of expressing the unknown vector b or the like in computation of an approximate value of an external magnetic field is different.

### Method of Computing Approximate Value of External Magnetic Field

In the second embodiment, in computation of an approximate value of an external magnetic field, the unknown vector b (first vector b) expressed by Equation (47) is defined on the basis of 21 unknowns aᵢⱼ, and the unknown vector b is solved by using Equation (51) or (52). In contrast, in the third embodiment, there is a difference in which the unknown aᵢⱼ is solved by applying the second equation of Maxwell's equations to the magnetic field B.

The second equation of Maxwell's equations corresponds to a Gauss' law regarding a magnetic field, and indicates that divergence of the magnetic field is zero. The second equation of Maxwell's equations is expressed by Equation (55). A relationship shown in Equation (55) is applied to Equation (20), Equation (21), Equation (23), or the like.

$$div \vec{B} = {\sum }_{i = 1}^{B} {\partial }_{i} {B}_{i} = \frac{\partial {B}_{1}}{\partial {r}_{1}} + \frac{\partial {B}_{2}}{\partial {r}_{2}} + \frac{\partial {B}_{3}}{\partial {r}_{3}} = \frac{\partial {B}_{x}}{\partial x} + \frac{\partial {B}_{y}}{\partial y} + \frac{\partial {B}_{z}}{\partial z} \qquad ( 55 )$$

If Equation (55) is assigned to Equation (20), Equation (56) is obtained.

divB̅=(a₁₂+a₁₅y+a₁₇z)+(a₂₃+a₂₅y+a₂₆z)+(a₃₄+a₃₆y+a₃₇x)=0 (56)

On the right side of Equation (56), the first parenthesis relates to the partial differentiation regarding x, the second parenthesis relates to the partial differentiation regarding y, and the third parenthesis relates to the partial differentiation regarding z. In the Gauss' law regarding a magnetic field, Equation (56) should be always zero. Therefore, the constant term, the proportional term regarding x, the proportional term regarding y, and the proportional term regarding z should all be zero on the right side of Equation (56). Thus, Equation (57) is obtained.

Equation (57) includes four identical equations, and thus the number of the unknowns aᵢⱼ is reduced from 21 to 17 (=21−4). Specifically, when the 21 unknowns aᵢⱼ are solved, Equation (58) is applied.

a₃₄=−(a₁₂+a₂₃)

a₃₇=−a₂₅

a₃₆=−a₁₅

a₂₆=−a₁₇ (58)

Four unknowns on the respective left sides of Equation (58) are not required to be solved, and, thus, in the present embodiment, an unknown vector c (also referred to as a third vector c) expressed by Equation (59) is defined and is solved.

$$\vec{c} ≡ ( \begin{matrix}{a}_{11} \\ {a}_{12} \\ {a}_{13} \\ {a}_{14} \\ {a}_{15} \\ {a}_{16} \\ {a}_{17} \\ {a}_{21} \\ {a}_{22} \\ {a}_{23} \\ {a}_{24} \\ {a}_{25} \\ {a}_{27} \\ {a}_{31} \\ {a}_{32} \\ {a}_{33} \\ {a}_{35}\end{matrix} ) \qquad ( 59 )$$

As shown in Equation (59), the unknown vector c (third vector c) is a column vector of seventeen rows and one column obtained by arranging 17 unknowns in one column except for four unknowns such as a₂₆, a₃₄, a₃₆, and a₃₇ among the 21 unknowns aᵢⱼ. A magnetic sensor term matrix S (fifth matrix S) corresponding to the third vector c is defined in Equation (60).

$$S ≡ ( \begin{matrix}{R}_{11} & {R}_{21} & {R}_{31} & {R}_{41} & {R}_{51} & {R}_{61} & {R}_{71} & 0 & 0 & 0 & 0 & 0 & 0 & 0 & 0 & 0 & 0 \\ 0 & 0 & 0 & 0 & 0 & 0 & - {R}_{61} & {R}_{11} & {R}_{21} & {R}_{31} & {R}_{41} & {R}_{51} & {R}_{71} & 0 & 0 & 0 & 0 \\ 0 & - {R}_{41} & 0 & 0 & - {R}_{61} & 0 & 0 & 0 & 0 & - {R}_{41} & 0 & - {R}_{71} & 0 & {R}_{11} & {R}_{21} & {R}_{31} & {R}_{51} \\ {R}_{21} & {R}_{22} & {R}_{32} & {R}_{42} & {R}_{52} & {R}_{62} & {R}_{72} & 0 & 0 & 0 & 0 & 0 & 0 & 0 & 0 & 0 & 0 \\ 0 & 0 & 0 & 0 & 0 & 0 & - {R}_{62} & {R}_{12} & {R}_{22} & {R}_{32} & {R}_{42} & {R}_{52} & {R}_{72} & 0 & 0 & 0 & 0 \\ 0 & - {R}_{42} & 0 & 0 & - {R}_{62} & 0 & 0 & 0 & 0 & - {R}_{42} & 0 & - {R}_{72} & 0 & {R}_{12} & {R}_{22} & {R}_{32} & {R}_{52} \\ 0 & 0 & 0 & 0 & 0 & 0 & - {R}_{63} & {R}_{13} & {R}_{23} & {R}_{33} & {R}_{43} & {R}_{53} & {R}_{73} & 0 & 0 & 0 & 0 \\ 0 & - {R}_{43} & 0 & 0 & - {R}_{63} & 0 & 0 & 0 & 0 & - {R}_{13} & 0 & - {R}_{23} & 0 & {R}_{13} & {R}_{23} & {R}_{33} & {R}_{53} \\ {R}_{14} & {R}_{24} & {R}_{34} & {R}_{44} & {R}_{54} & {R}_{64} & {R}_{74} & 0 & 0 & 0 & 0 & 0 & 0 & 0 & 0 & 0 & 0 \\ 0 & 0 & 0 & 0 & 0 & 0 & - {R}_{64} & {R}_{14} & {R}_{24} & {R}_{34} & {R}_{44} & {R}_{54} & {R}_{74} & 0 & 0 & 0 & 0 \\ 0 & - {R}_{44} & 0 & 0 & - {R}_{64} & 0 & 0 & 0 & 0 & - {R}_{44} & 0 & - {R}_{74} & 0 & {R}_{14} & {R}_{24} & {R}_{34} & {R}_{54} \\ ⋮ & & & & & & & & & & & & & & & & ⋮ \\ ⋮ & & & & & & & & & & & & & & & & ⋮ \\ ⋮ & & & & & & & & & & & & & & & & ⋮ \\ {R}_{1 k} & {R}_{2 k} & {R}_{3 k} & {R}_{4 k} & {R}_{5 k} & {R}_{6 k} & {R}_{7 k} & 0 & 0 & 0 & 0 & 0 & 0 & 0 & 0 & 0 & 0 \\ 0 & 0 & 0 & 0 & 0 & 0 & - {R}_{6 k} & {R}_{1 k} & {R}_{2 k} & {R}_{3 k} & {R}_{4 k} & {R}_{5 k} & {R}_{7 k} & 0 & 0 & 0 & 0 \\ 0 & - {R}_{1 k} & 0 & 0 & - {R}_{6 k} & 0 & 0 & 0 & 0 & - {R}_{4 k} & 0 & - {R}_{7 k} & 0 & {R}_{1 k} & {R}_{2 k} & {R}_{3 k} & {R}_{5 k} \\ ⋮ & & & & & & & & & & & & & & & & ⋮ \\ ⋮ & & & & & & & & & & & & & & & & ⋮ \\ ⋮ & & & & & & & & & & & & & & & & ⋮ \\ {R}_{1 α} & {R}_{2 α} & {R}_{3 α} & {R}_{4 α} & {R}_{5 α} & {R}_{6 α} & {R}_{7 α} & 0 & 0 & 0 & 0 & 0 & 0 & 0 & 0 & 0 & 0 \\ 0 & 0 & 0 & 0 & 0 & 0 & - {R}_{6 α} & {R}_{1 α} & {R}_{2 α} & {R}_{3 α} & {R}_{4 α} & {R}_{5 α} & {R}_{7 α} & 0 & 0 & 0 & 0 \\ 0 & 0 & 0 & 0 & 0 & 0 & - {R}_{6 a} & {R}_{61} & {R}_{2 a} & {R}_{3 a} & {R}_{4 a} & {R}_{5 a} & {R}_{7 a} & 0 & 0 & 0 & 0 \\ 0 & - {R}_{4 a} & 0 & 0 & - {R}_{6 a} & 0 & 0 & 0 & 0 & - {R}_{4 a} & 0 & - {R}_{7 a} & 0 & {R}_{1 a} & {R}_{2 a} & {R}_{3 a} & {R}_{4 a}\end{matrix} ) \qquad ( 60 )$$

As shown in Equation (60), the magnetic sensor term matrix S (fifth matrix S) is a matrix of 3α rows and seventeen columns. A single magnetic sensor term vector Rₖ corresponds to three rows of 3α rows. Specifically, components corresponding to the magnetic sensor term vector Rₖ at a k-th magnetic sensor position rₖ appear in a (3k−2)-th row, a (3k−1)-th row, and a 3k-th row of the fifth matrix S. The (3k−2)-th row of the fifth matrix S is used to obtain a first row component Bᵢₖ of a magnetic field detected by the k-th noise magnetic sensor 30.

Similarly, the (3k−1)-th row of the fifth matrix S is used to obtain a second row component B₂ₖ of the magnetic field detected by the k-th noise magnetic sensor 30, and the 3k-th row of the fifth matrix S is used to obtain a third row component B₃ₖ of the magnetic field detected by the k-th noise magnetic sensor 30. If the (3k−2)-th row of the fifth matrix S, (3k−1)-th row of the fifth matrix S, and the 3k-th row of the fifth matrix S are arranged in a partial matrix Tₖ of three rows and seventeen columns, the partial matrix Tₖ is expressed by Equation (61).

$$\begin{matrix}{T}_{k} = ( \begin{matrix}{R}_{11} & {R}_{21} & {R}_{31} & {R}_{41} & {R}_{51} & {R}_{61} & {R}_{71} & 0 & 0 & 0 & 0 & 0 & 0 & 0 & 0 & 0 & 0 \\ 0 & 0 & 0 & 0 & 0 & 0 & - {R}_{6 k} & {R}_{1 k} & {R}_{2 k} & {R}_{3 k} & {R}_{4 k} & {R}_{5 k} & {R}_{7 k} & 0 & 0 & 0 & 0 \\ 0 & - {R}_{1 k} & 0 & 0 & - {R}_{6 k} & 0 & 0 & 0 & 0 & - {R}_{4 k} & 0 & - {R}_{7 k} & 0 & {R}_{1 k} & {R}_{2 k} & {R}_{3 k} & {R}_{4 k}\end{matrix} ) \\ = ( \begin{matrix}1 & {x}_{k} & {y}_{k} & {z}_{k} & {x}_{k} {y}_{k} & {y}_{k} {z}_{k} & {z}_{k} {x}_{k} & 0 & 0 & 0 & 0 & 0 & 0 & 0 & 0 & 0 & 0 \\ 0 & 0 & 0 & 0 & 0 & 0 & - {y}_{k} {z}_{k} & 1 & {x}_{k} & {y}_{k} & {z}_{k} & {x}_{k} {y}_{k} & {z}_{k} {x}_{k} & 0 & 0 & 0 & 0 \\ 0 & - {z}_{k} & 0 & 0 & - {y}_{k} {z}_{k} & 0 & 0 & 0 & 0 & - {z}_{k} & 0 & - {z}_{k} {x}_{k} & 0 & 1 & {x}_{k} & {y}_{k} & {x}_{k} {y}_{k}\end{matrix} )\end{matrix} \qquad ( 61 )$$

In a case of using the partial matrix Tₖ, the fifth matrix S is a matrix obtained by arranging a partial matrices including the partial matrix T₁ of k=1 to the partial matrix T_{α} of k=α in α rows and one column, and is expressed by Equation (62).

$$S = ( \begin{matrix}{T}_{1} \\ {T}_{2} \\ {T}_{3} \\ ⋮ \\ {T}_{a}\end{matrix} ) \qquad ( 62 )$$

The detection magnetic field vector N is expressed by Equation (63) by using the unknown vector c (third vector c) defined in Equation (59) and the magnetic sensor term matrix S (fifth matrix S) defined in Equation (60).

$$\vec{N} = S \vec{c} , ( \begin{matrix}{B}_{11} \\ {B}_{21} \\ {B}_{31} \\ {B}_{12} \\ {B}_{22} \\ {B}_{32} \\ ⋮ \\ {B}_{1 α - 1} \\ {B}_{1 α} \\ {B}_{2 α} \\ {B}_{3 α}\end{matrix} ) = ( \begin{matrix}1 & {x}_{1} & {y}_{1} & {z}_{1} & {x}_{1} {y}_{1} & {y}_{1} {z}_{1} & {z}_{1} {x}_{1} & 0 & 0 & 0 & 0 & 0 & 0 & 0 & 0 & 0 & 0 \\ 0 & 0 & 0 & 0 & 0 & 0 & - {y}_{1} {z}_{1} & 1 & {x}_{1} & {y}_{1} & {z}_{1} & {x}_{1} {y}_{1} & {z}_{1} {x}_{1} & 0 & 0 & 0 & 0 \\ 0 & - {z}_{1} & 0 & 0 & - {y}_{1} {z}_{1} & 0 & 0 & 0 & 0 & - {z}_{1} & 0 & - {z}_{1} {x}_{1} & 0 & 1 & {x}_{1} & {y}_{1} & {x}_{1} {y}_{1} \\ 1 & {x}_{2} & {y}_{2} & {z}_{2} & {x}_{2} {y}_{2} & {y}_{2} {z}_{2} & {z}_{2} {x}_{2} & 0 & 0 & 0 & 0 & 0 & 0 & 0 & 0 & 0 & 0 \\ 0 & 0 & 0 & 0 & 0 & 0 & - {y}_{2} {z}_{2} & 1 & {x}_{2} & {y}_{2} & {z}_{2} & {x}_{2} {y}_{2} & {z}_{2} {x}_{2} & 0 & 0 & 0 & 0 \\ 0 & - {z}_{2} & 0 & 0 & - {y}_{2} {z}_{2} & 0 & 0 & 0 & 0 & - {z}_{2} & - & - {z}_{2} {x}_{2} & 0 & 1 & {x}_{2} & {y}_{2} & {x}_{2} {y}_{2} \\ ⋮ & & & & & & & & & & & & & & & & ⋮ \\ 0 & - {z}_{α - 1} & 0 & 0 & - {y}_{α - 1} {z}_{α - 1} & 0 & 0 & 0 & 0 & - {z}_{α - 1} & 0 & - {z}_{a - 1} {x}_{α - 1} & 0 & 1 & {x}_{α - 1} & {y}_{α - 1} & {x}_{α - 1} {y}_{α - 1} \\ 1 & {x}_{α} & {y}_{α} & {z}_{α} & {x}_{α} {y}_{α} & {y}_{α} {z}_{α} & {z}_{α} {x}_{α} & 0 & 0 & 0 & 0 & 0 & 0 & 0 & 0 & 0 & 0 \\ 0 & 0 & 0 & 0 & 0 & 0 & - {y}_{α} {z}_{α} & 1 & {x}_{α} & {y}_{α} & {z}_{α} & {x}_{α} {y}_{α} & {z}_{α} {x}_{α} & 0 & 0 & 0 & 0 \\ 0 & - {z}_{α} & 0 & 0 & - {y}_{α} {z}_{α} & 0 & 0 & 0 & 0 & - {z}_{α} & 0 & - {z}_{α} {x}_{α} & 0 & 1 & {x}_{α} & {y}_{α} & {x}_{α} {y}_{α}\end{matrix} ) ( \begin{matrix}{a}_{11} \\ {a}_{12} \\ {a}_{13} \\ {a}_{14} \\ {a}_{15} \\ {a}_{16} \\ {a}_{17} \\ {a}_{21} \\ {a}_{22} \\ {a}_{23} \\ {a}_{24} \\ {a}_{25} \\ {a}_{27} \\ {a}_{31} \\ {a}_{32} \\ {a}_{33} \\ {a}_{35}\end{matrix} ) \qquad ( 63 )$$

Here, in the same manner as in the second embodiment, if Equation (63) is expressed by using matrix elements in consideration of Equation (58) expressing the second Equation of Maxwell's equations, this leads to Equation (32). Therefore, it can be seen that Equation (63) represents the same equation system as in the first embodiment or the second embodiment.

The unknown vector c is to be obtained from Equation (63). If the number α of noise magnetic sensors is 6 or larger, the number of rows of the detection magnetic field vector N (second vector N) is 18 or larger in relation to the number 17 of unknowns, and thus the unknowns can be specified by using a least square method. In this case, the magnetic sensor term matrix S (fifth matrix S) is not a square matrix, and thus an inverse matrix thereof is not present. In this case, as shown in Equation (64), the unknown vector c (third vector c) is obtained by multiplying the detection magnetic field vector N (second vector N) by a pseudo-inverse matrix (also referred to as a generalized inverse matrix) S⁺ of the magnetic sensor term matrix S (fifth matrix S) from the left.

c̅=S⁺N̅ (64)

In Equation (64), the pseudo-inverse matrix S⁺ of the fifth matrix S is obtained by using Equation (65).

S⁺=(S^{T}S)⁻¹S^{T} (65)

As shown in Equation (65), the pseudo-inverse matrix S⁺ is obtained by multiplying an inverse matrix of a product between a transposed matrix S^{T} of the fifth matrix S and the fifth matrix S by the transposed matrix S^{T} of the fifth matrix S. The transposed matrix S^{T} of the fifth matrix S is obtained by replacing matrix elements of the fifth matrix S with respect to rows and columns.

If the pseudo-inverse matrix S⁺ is used to obtain the unknown vector c, a principle of the least square method acts, and thus an optimal solution which minimizes errors is defined. As mentioned above, in the third embodiment, since the second equation of Maxwell's equations is taken into consideration, it is possible to specify a magnetic field in the measurement target space by using a smaller number of the noise magnetic sensors 30 than in the second embodiment. In a case of using the same number of noise magnetic sensors 30 as in the second embodiment, since the number of unknowns is reduced by four, a more appropriate solution than the optimal solution obtained in the second embodiment can be obtained, and thus it is possible to more accurately approximate a magnetic field in the measurement target space.

In the present embodiment, a description has been made of an example in which the result (Equation (58)) of the Gauss' law regarding a magnetic field is applied to the second embodiment, but the result (Equation (58)) of the Gauss' law regarding a magnetic field may be applied to the first embodiment.

Next, a description will be made of a method of computing the magnetic sensor term matrix S (fifth matrix S) in computation of an approximate value of an external magnetic field in the third embodiment by using Examples of arrangement of the noise magnetic sensors 30.

### Example 3-1

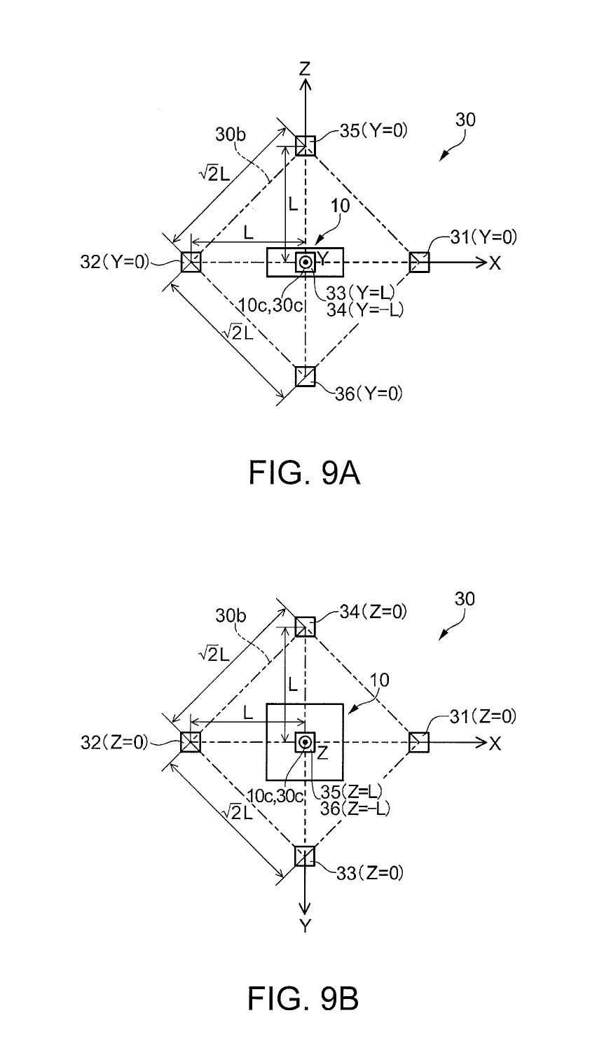

FIGS. 8A to 9B are diagrams illustrating arrangement of the noise magnetic sensors related to Example 3-1. Specifically, FIG. 8A is a perspective view, and FIG. 8B is a plan view which is viewed from the +X direction side in FIG. 8A. FIG. 9A is a plan view which is viewed from the +Y direction side in FIG. 8A, and FIG. 9B is a plan view which is viewed from the +Z direction side in FIG. 8A.

In Example 3-1, as illustrated in FIG. 8A, two of six noise magnetic sensors 30 are disposed on each of the X axis, the Y axis, and the Z axis so as to be symmetric to each other with respect to the origin. Therefore, in Example 3-1, the six noise magnetic sensors 30 are respectively disposed at vertices of a regular octahedron 30b of which a length of one side is 2^{1/2}×L. The center 30c of the regular octahedron 30b substantially matches the center 10c of the heart magnetic field sensor 10.

In Example 3-1, as illustrated in FIGS. 8A and 8B, among the six noise magnetic sensors 30, four sensors such as the third noise magnetic sensor 33, the fourth noise magnetic sensor 34, the fifth noise magnetic sensor 35, and the sixth noise magnetic sensor 36 are disposed in a plane of X=0 parallel to the Y-Z plane.

As illustrated in FIGS. 8A and 9A, four sensors such as the first noise magnetic sensor 31, the second noise magnetic sensor 32, the fifth noise magnetic sensor 35, and the sixth noise magnetic sensor 36 are disposed in a plane of Y=0 parallel to the X-Z plane. As illustrated in FIGS. 8A and 9B, four sensors such as the first noise magnetic sensor 31, the second noise magnetic sensor 32, the third noise magnetic sensor 33, and the fourth noise magnetic sensor 34 are disposed in a plane of Z=0 parallel to the X-Y plane.

Among a line segment connecting the first noise magnetic sensor 31 to the second noise magnetic sensor 32, a line segment connecting the third noise magnetic sensor 33 to the fourth noise magnetic sensor 34, and a line segment connecting the fifth noise magnetic sensor 35 to the sixth noise magnetic sensor 36, at least two line segments are orthogonal to each other. The heart magnetic field sensor 10 is disposed so that the remaining line segment intersects a plane which is parallel to the two line segments which are orthogonal to each other. The heart magnetic field sensor 10 is disposed at a position including an intersection portion at which the remaining line segment intersects the plane which is parallel to the two line segments orthogonal to each other.

A position vector rₖ (magnetic sensor position rₖ) of the noise magnetic sensors 30 disposed in the above-described manner is expressed by Equation (66).

$${\vec{r}}_{1} = L ( \begin{matrix}1 \\ 0 \\ 0\end{matrix} ) {\vec{r}}_{2} = L ( \begin{matrix}- 1 \\ 0 \\ 0\end{matrix} ) {\vec{r}}_{3} = L ( \begin{matrix}0 \\ 1 \\ 0\end{matrix} ) {\vec{r}}_{4} = L ( \begin{matrix}0 \\ - 1 \\ 0\end{matrix} ) {\vec{r}}_{5} = L ( \begin{matrix}0 \\ 0 \\ 1\end{matrix} ) {\vec{r}}_{6} = L ( \begin{matrix}0 \\ 0 \\ - 1\end{matrix} ) \qquad ( 66 )$$

If the six noise magnetic sensors 30 are disposed in the above-described manner, the magnetic sensor term matrix S (fifth matrix S) is expressed with 1, L, and −L as shown in Equation (67), and the number of zero matrix elements increases. Thus, computation is further simplified.

$$S = ( \begin{matrix}1 & L & 0 & 0 & 0 & 0 & 0 & 0 & 0 & 0 & 0 & 0 & 0 & 0 & 0 & 0 & 0 \\ 0 & 0 & 0 & 0 & 0 & 0 & 1 & L & 0 & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & L & 0 & 0 \\ 1 & - L & 0 & 0 & 0 & 0 & 0 & 0 & 0 & 0 & 0 & 0 & 0 & 0 & 0 & 0 & 0 \\ 0 & 0 & 0 & 0 & 0 & 0 & 1 & - L & 0 & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & - L & 0 & 0 \\ 1 & 0 & L & 0 & 0 & 0 & 0 & 0 & 0 & 0 & 0 & 0 & 0 & 0 & 0 & 0 & 0 \\ 0 & 0 & 0 & 0 & 0 & 0 & 1 & 0 & L & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & 0 & L & 0 \\ 1 & 0 & - L & 0 & 0 & 0 & 0 & 0 & 0 & 0 & 0 & 0 & 0 & 0 & 0 & 0 & 0 \\ 0 & 0 & 0 & 0 & 0 & 0 & 1 & 0 & - L & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & 0 & - L & 0 \\ 1 & 0 & 0 & L & 0 & 0 & 0 & 0 & 0 & 0 & 0 & 0 & 0 & 0 & 0 & 0 & 0 \\ 0 & 0 & 0 & 0 & 0 & 0 & 1 & 0 & 0 & L & 0 & 0 & 0 & 0 & 0 & 0 & 0 \\ 0 & - L & 0 & 0 & 0 & 0 & 0 & 0 & 0 & - L & 0 & 0 & 0 & 1 & 0 & 0 & 0 \\ 1 & 0 & 0 & - L & 0 & 0 & 0 & 0 & 0 & 0 & 0 & 0 & 0 & 0 & 0 & 0 & 0 \\ 0 & 0 & 0 & 0 & 0 & 0 & 1 & 0 & 0 & - L & 0 & 0 & 0 & 0 & 0 & 0 & 0 \\ 0 & L & 0 & 0 & 0 & 0 & 0 & 0 & 0 & L & 0 & 0 & 0 & 1 & 0 & 0 & 0\end{matrix} ) \qquad ( 67 )$$

If L is used as one unit of the XYZ coordinate system, the magnetic sensor term matrix S (fifth matrix S) is expressed with 1 and −1 as shown in Equation (68), and thus computation is still further simplified.

$$S = ( \begin{matrix}1 & 1 & 0 & 0 & 0 & 0 & 0 & 0 & 0 & 0 & 0 & 0 & 0 & 0 & 0 & 0 & 0 \\ 0 & 0 & 0 & 0 & 0 & 0 & 1 & 1 & 0 & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & 1 & 0 & 0 \\ 1 & - 1 & 0 & 0 & 0 & 0 & 0 & 0 & 0 & 0 & 0 & 0 & 0 & 0 & 0 & 0 & 0 \\ 0 & 0 & 0 & 0 & 0 & 0 & 1 & - 1 & 0 & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & - 1 & 0 & 0 \\ 1 & 0 & L & 0 & 0 & 0 & 0 & 0 & 0 & 0 & 0 & 0 & 0 & 0 & 0 & 0 & 0 \\ 0 & 0 & 0 & 0 & 0 & 0 & 1 & 0 & 1 & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & 0 & 1 & 0 \\ 1 & 0 & - 1 & 0 & 0 & 0 & 0 & 0 & 0 & 0 & 0 & 0 & 0 & 0 & 0 & 0 & 0 \\ 0 & 0 & 0 & 0 & 0 & 0 & 1 & 0 & - 1 & 0 & 0 & 0 & 0 & 0 & 0 & 0 & 0 \\ 0 & 0 & 0 & 0 & 0 & 0 & 0 & 0 & 0 & 0 & 0 & 0 & 0 & 1 & 0 & - 1 & 0 \\ 1 & 0 & 0 & 1 & 0 & 0 & 0 & 0 & 0 & 0 & 0 & 0 & 0 & 0 & 0 & 0 & 0 \\ 0 & 0 & 0 & 0 & 0 & 0 & 1 & 0 & 0 & 1 & 0 & 0 & 0 & 0 & 0 & 0 & 0 \\ 0 & - 1 & 0 & 0 & 0 & 0 & 0 & 0 & 0 & - 1 & 0 & 0 & 0 & 1 & 0 & 0 & 0 \\ 1 & 0 & 0 & - 1 & 0 & 0 & 0 & 0 & 0 & 0 & 0 & 0 & 0 & 0 & 0 & 0 & 0 \\ 0 & 0 & 0 & 0 & 0 & 0 & 1 & 0 & 0 & - 1 & 0 & 0 & 0 & 0 & 0 & 0 & 0 \\ 0 & 1 & 0 & 0 & 0 & 0 & 0 & 0 & 0 & 1 & 0 & 0 & 0 & 1 & 0 & 0 & 0\end{matrix} ) \qquad ( 68 )$$

The above-described embodiments are only an aspect of the invention, and may be arbitrarily modified and altered within the scope of the invention. For example, the following modification example may be considered.

### Modification Example

In the above-described embodiments, the magnetic field B is approximated by using a polynomial which includes three variables and is a polynomial of a quadratic expression regarding the variables, shown in Equation (20), but an approximate expression of the magnetic field B is not limited to Equation (20). For example, in a case where an external magnetic field spatially includes a high-order gradient magnetic field, a higher-order term may be added to Equation (20). In this case, since a larger number of noise magnetic sensors 30 than the number of the coefficients aᵢⱼ as unknowns are necessary, the number of noise magnetic sensors 30 to be disposed increases as the number of the coefficients aᵢⱼ increases, and thus the size of the unknown matrix a (first matrix a) also increases.

The entire disclosure of Japanese Patent Application No. 2015-104275, filed May 22, 2015 is expressly incorporated by reference herein.

What is claimed is:

1. A magnetic measurement system comprising:
a first magnetic sensor that measures a first magnetic field and a second magnetic field;
a second magnetic sensor that measures the second magnetic field; and
a processing apparatus that computes an approximate value of the second magnetic field in the first magnetic sensor by using a measurement value in the second magnetic sensor and a multi-variable polynomial.

2. A magnetic measurement system comprising:
a first magnetic sensor that measures a first magnetic field and a second magnetic field;
a second magnetic sensor that measures the second magnetic field; and
a processing apparatus that computes an approximate value of the second magnetic field in the first magnetic sensor by using a measurement value in the second magnetic sensor and a non-linear polynomial.

3. The magnetic measurement system according to claim 1,
wherein the processing apparatus subtracts the approximate value of the second magnetic field from a measurement value in the first magnetic sensor.

4. The magnetic measurement system according to claim 2,
wherein the processing apparatus subtracts the approximate value of the second magnetic field from a measurement value in the first magnetic sensor.

5. The magnetic measurement system according to claim 1,
wherein the multi-variable polynomial is expressed by Equation (1):
Bᵢ=aᵢ₁+aᵢ₂x+aᵢ₃y+aᵢ₄z+aᵢ₅xy+aᵢ₆yz+aᵢ₇zx (1)
in Equation (1), aᵢⱼ, (where i is an integer of 1 to 3, and j is an integer of 1 to 7) is a coefficient, x, y, and z are space coordinates of an approximate value B of a magnetic field, and Bᵢ is an i-th component of the approximate value B of the magnetic field.

6. The magnetic measurement system according to claim 2,
wherein the non-linear polynomial is expressed by the above Equation (1).

7. The magnetic measurement system according to claim 1,
wherein a solution of the multi-variable polynomial is obtained by using a least square method on the basis of the measurement value in the second magnetic sensor.

8. The magnetic measurement system according to claim 2,
wherein a solution of the non-linear polynomial is obtained by using a least square method on the basis of the measurement value in the second magnetic sensor.

9. The magnetic measurement system according to claim 5,
wherein the second magnetic sensor measures 21 or more magnetic field vector components of the second magnetic field.

10. The magnetic measurement system according to claim 6,
wherein the second magnetic sensor measures 21 or more magnetic field vector components of the second magnetic field.

11. The magnetic measurement system according to claim 9,
wherein, when a first matrix formed of unknowns of the above Equation (1) is indicated by a which is expressed by Equation (2), a second matrix formed of the measurement value in the second magnetic sensor is indicated by M which is expressed by Equation (3), and a third matrix formed of a position of the second magnetic sensor is indicated by P which is expressed by Equation (4), the first matrix a is obtained by using Equation (5) or Equation (6):
$$\begin{matrix}\vec{B} ( \vec{r} ) = ( \begin{matrix}{B}_{x} \\ {B}_{y} \\ {B}_{z}\end{matrix} ) = ( \begin{matrix}{a}_{11} & {a}_{12} & {a}_{13} & {a}_{14} & {a}_{15} & {a}_{16} & {a}_{17} \\ {a}_{21} & {a}_{22} & {a}_{23} & {a}_{24} & {a}_{25} & {a}_{26} & {a}_{27} \\ {a}_{31} & {a}_{32} & {a}_{33} & {a}_{34} & {a}_{35} & {a}_{36} & {a}_{37}\end{matrix} ) ( \begin{matrix}1 \\ x \\ y \\ z \\ xy \\ yz \\ zx\end{matrix} ) ≡ a \vec{R} & ( 2 ) \\ M = ( {\vec{B}}_{1} ⋯ {\vec{B}}_{α} ) = ( \begin{matrix}{B}_{11} & {B}_{21} & … & {B}_{1 α} \\ {B}_{21} & {B}_{22} & … & {B}_{2 α} \\ {B}_{31} & {B}_{32} & … & {B}_{3 α}\end{matrix} ) & ( 3 ) \\ P = ( {\vec{R}}_{1} ⋯ {\vec{R}}_{α} ) = ( \begin{matrix}{R}_{11} & {R}_{12} & … & {R}_{1 α} \\ {R}_{21} & {R}_{22} & … & {R}_{2 α} \\ {R}_{31} & {R}_{32} & … & {R}_{3 α} \\ {R}_{41} & {R}_{42} & … & {R}_{4 α} \\ {R}_{51} & {R}_{52} & … & {R}_{5 α} \\ {R}_{61} & {R}_{62} & … & {R}_{6 α} \\ {R}_{71} & {R}_{62} & … & {R}_{7 α}\end{matrix} ) & ( 4 ) \\ a = {MP}^{- 1} & ( 5 ) \\ a = {MP}^{+} & ( 6 )\end{matrix}$$
in Equation (5), P⁻¹ is an inverse matrix of the third matrix P, and, in Equation (6), P⁺ is a pseudo-inverse matrix of the third matrix P.

12. The magnetic measurement system according to claim 9,
wherein, when a first vector formed of unknowns of the above Equation (1) is indicated by b which is expressed by Equation (7), a second vector formed of the measurement value in the second magnetic sensor is indicated by N which is expressed by Equation (8), and a fourth matrix formed of a position of the second magnetic sensor is indicated by Q which is expressed by Equation (9), the first vector b is obtained by using Equation (10) or Equation (11):
$$\begin{matrix}\vec{b} ≡ ( \begin{matrix}{a}_{11} \\ {a}_{12} \\ ⋮ \\ {a}_{16} \\ {a}_{17} \\ {a}_{21} \\ ⋮ \\ {a}_{27} \\ {a}_{31} \\ ⋮ \\ {a}_{37}\end{matrix} ) = ( \begin{matrix}{b}_{1} \\ {b}_{2} \\ ⋮ \\ {b}_{6} \\ {b}_{7} \\ {b}_{8} \\ ⋮ \\ {b}_{14} \\ {b}_{15} \\ ⋮ \\ {b}_{21}\end{matrix} ) & ( 7 ) \\ \vec{N} ≡ ( \begin{matrix}{B}_{11} \\ {B}_{21} \\ {B}_{31} \\ {B}_{12} \\ {B}_{22} \\ {B}_{32} \\ ⋮ \\ {B}_{3 α - 1} \\ {B}_{1 α} \\ {B}_{2 α} \\ {B}_{3 α}\end{matrix} ) = ( \begin{matrix}{n}_{1} \\ {n}_{2} \\ {n}_{3} \\ {n}_{4} \\ {n}_{5} \\ {n}_{6} \\ ⋮ \\ {n}_{3 α - 1} \\ {n}_{3 α - 2} \\ {n}_{3 α - 1} \\ {n}_{3 α}\end{matrix} ) & ( 8 ) \\ \begin{matrix}Q ≡ ( \begin{matrix}{\vec{R}}_{1}^{T} & \vec{0} & \vec{0} \\ \vec{0} & {\vec{R}}_{1}^{T} & \vec{0} \\ \vec{0} & \vec{0} & {\vec{R}}_{1}^{T} \\ {\vec{R}}_{2}^{T} & \vec{0} & \vec{0} \\ \vec{0} & {\vec{R}}_{2}^{T} & \vec{0} \\ \vec{0} & \vec{0} & {\vec{R}}_{2}^{T} \\ \vec{0} & \vec{0} & {\vec{R}}_{a - 1}^{T} \\ {\vec{R}}_{a}^{T} & \vec{0} & \vec{0} \\ \vec{0} & {\vec{R}}_{a}^{T} & \vec{0} \\ \vec{0} & \vec{0} & {\vec{R}}_{a}^{T}\end{matrix} ) \\ = ( \begin{matrix}{R}_{11} & {R}_{12} & ⋯ & {R}_{61} & {R}_{71} & 0 & 0 & ⋯ & 0 & 0 & 0 & 0 & ⋯ & 0 & 0 \\ 0 & 0 & ⋯ & 0 & 0 & {R}_{11} & {R}_{21} & ⋯ & {R}_{61} & {R}_{71} & 0 & 0 & ⋯ & 0 & 0 \\ 0 & 0 & ⋯ & 0 & 0 & 0 & 0 & ⋯ & 0 & 0 & {R}_{11} & {R}_{21} & ⋯ & {R}_{61} & {R}_{71} \\ {R}_{12} & {R}_{22} & ⋯ & {R}_{62} & {R}_{72} & 0 & 0 & ⋯ & 0 & 0 & 0 & 0 & ⋯ & 0 & 0 \\ 0 & 0 & ⋯ & 0 & 0 & {R}_{12} & {R}_{22} & ⋯ & {R}_{62} & {R}_{72} & 0 & 0 & ⋯ & 0 & 0 \\ 0 & 0 & ⋯ & 0 & 0 & 0 & 0 & ⋯ & 0 & 0 & {R}_{12} & {R}_{22} & ⋯ & {R}_{62} & {R}_{72} \\ ⋮ & ⋮ & & ⋮ & ⋮ & ⋮ & ⋮ & & ⋮ & ⋮ & ⋮ & ⋮ & & ⋮ & ⋮ \\ 0 & 0 & ⋯ & 0 & 0 & 0 & 0 & ⋯ & 0 & 0 & {R}_{1 a - 1} & {R}_{2 a - 1} & ⋯ & {R}_{6 a - 1} & {R}_{7 a - 1} \\ {R}_{1 a} & {R}_{2 a} & ⋯ & {R}_{6 a} & {R}_{7 a} & 0 & 0 & ⋯ & 0 & 0 & 0 & 0 & ⋯ & 0 & 0 \\ 0 & 0 & ⋯ & 0 & 0 & {R}_{1 a} & {R}_{2 a} & ⋯ & {R}_{6 a} & {R}_{7 a} & 0 & 0 & ⋯ & 0 & 0 \\ 0 & 0 & ⋯ & 0 & 0 & 0 & 0 & ⋯ & 0 & 0 & {R}_{1 a} & {R}_{2 a} & ⋯ & {R}_{6 a} & {R}_{7 a}\end{matrix} )\end{matrix} & ( 9 ) \\ \vec{b} = {Q}^{- 1} \vec{N} & ( 10 ) \\ \vec{b} = {Q}^{+} \vec{N} & ( 11 )\end{matrix}$$

13. The magnetic measurement system according to claim 5,
wherein the multi-variable polynomial is expressed by the above Equation (1) in consideration of Equation (12):
a₃₄=−(a₁₂+a₂₃)
a₃₇=−a₂₅
a₃₆=−a₁₅
a₂₆=−a₁₇ (12)

14. The magnetic measurement system according to claim 6,
wherein the non-linear polynomial is expressed by the above Equation (1) in consideration of the above Equation (12).

15. The magnetic measurement system according to claim 13,
wherein the second magnetic sensor measures 17 or more magnetic field vector components of the second magnetic field.

16. The magnetic measurement system according to claim 15,
wherein, when a third vector formed of unknowns of the above Equation (1) is indicated by c which is expressed by Equation (13), a second vector formed of the measurement value in the second magnetic sensor is indicated by N which is expressed by the above Equation (8), and a fifth matrix formed of a position of the second magnetic sensor is indicated by S which is expressed by Equations (14) and (15), or Equation (16), the third vector c is obtained by using Equation (17):
$$\begin{matrix}\vec{c} ≡ ( \begin{matrix}{a}_{11} \\ {a}_{12} \\ {a}_{13} \\ {a}_{14} \\ {a}_{15} \\ {a}_{16} \\ {a}_{17} \\ {a}_{21} \\ {a}_{22} \\ {a}_{23} \\ {a}_{24} \\ {a}_{25} \\ {a}_{27} \\ {a}_{31} \\ {a}_{32} \\ {a}_{33} \\ {a}_{35}\end{matrix} ) & ( 13 ) \\ S = ( \begin{matrix}{T}_{1} \\ {T}_{2} \\ {T}_{3} \\ ⋮ \\ {T}_{α}\end{matrix} ) & ( 14 ) \\ \begin{matrix}{T}_{k} = ( \begin{matrix}{R}_{11} & {R}_{21} & {R}_{31} & {R}_{41} & {R}_{51} & {R}_{61} & {R}_{71} & 0 & 0 & 0 & 0 & 0 & 0 & 0 & 0 & 0 & 0 \\ 0 & 0 & 0 & 0 & 0 & 0 & - {R}_{6 k} & {R}_{1 k} & {R}_{2 k} & {R}_{3 k} & {R}_{4 k} & {R}_{5 k} & {R}_{7 k} & 0 & 0 & 0 & 0 \\ 0 & - {R}_{1 k} & 0 & 0 & - {R}_{6 k} & 0 & 0 & 0 & 0 & - {R}_{4 k} & 0 & - {R}_{7 k} & 0 & {R}_{1 k} & {R}_{2 k} & {R}_{3 k} & {R}_{4 k}\end{matrix} ) \\ = ( \begin{matrix}1 & {x}_{k} & {y}_{k} & {z}_{k} & {x}_{k} {y}_{k} & {y}_{k} {z}_{k} & {z}_{k} {x}_{k} & 0 & 0 & 0 & 0 & 0 & 0 & 0 & 0 & 0 & 0 \\ 0 & 0 & 0 & 0 & 0 & 0 & - {y}_{k} {z}_{k} & 1 & {x}_{k} & {y}_{k} & {z}_{k} & {x}_{k} {y}_{k} & {z}_{k} {x}_{k} & 0 & 0 & 0 & 0 \\ 0 & - {z}_{k} & 0 & 0 & - {y}_{k} {z}_{k} & 0 & 0 & 0 & 0 & - {z}_{k} & 0 & - {z}_{k} {x}_{k} & 0 & 1 & {x}_{k} & {y}_{k} & {x}_{k} {y}_{k}\end{matrix} )\end{matrix} & ( 15 ) \\ S ≡ ( \begin{matrix}{R}_{11} & {R}_{21} & {R}_{31} & {R}_{41} & {R}_{51} & {R}_{61} & {R}_{71} & 0 & 0 & 0 & 0 & 0 & 0 & 0 & 0 & 0 & 0 \\ 0 & 0 & 0 & 0 & 0 & 0 & - {R}_{61} & {R}_{11} & {R}_{21} & {R}_{31} & {R}_{41} & {R}_{51} & {R}_{71} & 0 & 0 & 0 & 0 \\ 0 & - {R}_{41} & 0 & 0 & - {R}_{61} & 0 & 0 & 0 & 0 & - {R}_{41} & 0 & - {R}_{71} & 0 & {R}_{11} & {R}_{21} & {R}_{31} & {R}_{51} \\ {R}_{21} & {R}_{22} & {R}_{32} & {R}_{42} & {R}_{52} & {R}_{62} & {R}_{72} & 0 & 0 & 0 & 0 & 0 & 0 & 0 & 0 & 0 & 0 \\ 0 & 0 & 0 & 0 & 0 & 0 & - {R}_{62} & {R}_{12} & {R}_{22} & {R}_{32} & {R}_{42} & {R}_{52} & {R}_{72} & 0 & 0 & 0 & 0 \\ 0 & - {R}_{42} & 0 & 0 & - {R}_{62} & 0 & 0 & 0 & 0 & - {R}_{42} & 0 & - {R}_{72} & 0 & {R}_{12} & {R}_{22} & {R}_{32} & {R}_{52} \\ 0 & 0 & 0 & 0 & 0 & 0 & - {R}_{63} & {R}_{13} & {R}_{23} & {R}_{33} & {R}_{43} & {R}_{53} & {R}_{73} & 0 & 0 & 0 & 0 \\ 0 & - {R}_{43} & 0 & 0 & - {R}_{63} & 0 & 0 & 0 & 0 & - {R}_{13} & 0 & - {R}_{23} & 0 & {R}_{13} & {R}_{23} & {R}_{33} & {R}_{53} \\ {R}_{14} & {R}_{24} & {R}_{34} & {R}_{44} & {R}_{54} & {R}_{64} & {R}_{74} & 0 & 0 & 0 & 0 & 0 & 0 & 0 & 0 & 0 & 0 \\ 0 & 0 & 0 & 0 & 0 & 0 & - {R}_{64} & {R}_{14} & {R}_{24} & {R}_{34} & {R}_{44} & {R}_{54} & {R}_{74} & 0 & 0 & 0 & 0 \\ 0 & - {R}_{44} & 0 & 0 & - {R}_{64} & 0 & 0 & 0 & 0 & - {R}_{44} & 0 & - {R}_{74} & 0 & {R}_{14} & {R}_{24} & {R}_{34} & {R}_{54} \\ ⋮ & & & & & & & & & & & & & & & & ⋮ \\ ⋮ & & & & & & & & & & & & & & & & ⋮ \\ ⋮ & & & & & & & & & & & & & & & & ⋮ \\ {R}_{1 k} & {R}_{2 k} & {R}_{3 k} & {R}_{4 k} & {R}_{5 k} & {R}_{6 k} & {R}_{7 k} & 0 & 0 & 0 & 0 & 0 & 0 & 0 & 0 & 0 & 0 \\ 0 & 0 & 0 & 0 & 0 & 0 & - {R}_{6 k} & {R}_{1 k} & {R}_{2 k} & {R}_{3 k} & {R}_{4 k} & {R}_{5 k} & {R}_{7 k} & 0 & 0 & 0 & 0 \\ 0 & - {R}_{1 k} & 0 & 0 & - {R}_{6 k} & 0 & 0 & 0 & 0 & - {R}_{4 k} & 0 & - {R}_{7 k} & 0 & {R}_{1 k} & {R}_{2 k} & {R}_{3 k} & {R}_{5 k} \\ ⋮ & & & & & & & & & & & & & & & & ⋮ \\ ⋮ & & & & & & & & & & & & & & & & ⋮ \\ ⋮ & & & & & & & & & & & & & & & & ⋮ \\ {R}_{1 α} & {R}_{2 α} & {R}_{3 α} & {R}_{4 α} & {R}_{5 α} & {R}_{6 α} & {R}_{7 α} & 0 & 0 & 0 & 0 & 0 & 0 & 0 & 0 & 0 & 0 \\ 0 & 0 & 0 & 0 & 0 & 0 & - {R}_{6 α} & {R}_{1 α} & {R}_{2 α} & {R}_{3 α} & {R}_{4 α} & {R}_{5 α} & {R}_{7 α} & 0 & 0 & 0 & 0 \\ 0 & 0 & 0 & 0 & 0 & 0 & - {R}_{6 a} & {R}_{61} & {R}_{2 a} & {R}_{3 a} & {R}_{4 a} & {R}_{5 a} & {R}_{7 a} & 0 & 0 & 0 & 0 \\ 0 & - {R}_{4 a} & 0 & 0 & - {R}_{6 a} & 0 & 0 & 0 & 0 & - {R}_{4 a} & 0 & - {R}_{7 a} & 0 & {R}_{1 a} & {R}_{2 a} & {R}_{3 a} & {R}_{4 a}\end{matrix} ) & ( 16 ) \\ \vec{c} = {S}^{+} \vec{N} & ( 17 )\end{matrix}$$

17. A magnetic measurement system comprising:
a first magnetic sensor that measures a first magnetic field and a second magnetic field;
a first-second magnetic sensor and a second-second magnetic sensor that are disposed around the first magnetic sensor; and
a processing apparatus that computes an approximate value of the second magnetic field in the first magnetic sensor by using a measurement value in the first-second magnetic sensor and a measurement value in the second-second magnetic sensor,
wherein the first magnetic sensor is disposed at a position including the centroid between the first-second magnetic sensor and the second-second magnetic sensor.

18. The magnetic measurement system according to claim 17, further comprising:
a third-second magnetic sensor and a fourth-second magnetic sensor,
wherein the third-second magnetic sensor and the fourth-second magnetic sensor are disposed at positions which are symmetric to each other with respect to the centroid, and
wherein a line segment connecting the first-second magnetic sensor to the second-second magnetic sensor intersects a line segment connecting the third-second magnetic sensor to the fourth-second magnetic sensor.

19. The magnetic measurement system according to claim 18,
wherein a line segment connecting the first-second magnetic sensor to the second-second magnetic sensor is orthogonal to a line segment connecting the third-second magnetic sensor to the fourth-second magnetic sensor.
